(19) United States
(12) Patent Application Publication
(10) Pub. No.: US 2006/0015065 A1
(43) Pub. Date: Jan. 19, 2006

(54) Method of drug perfusion in paraaortic lymph node tumors, sheath for inserting catheter, and oxygenated blood perfusion apparatus

(76) Inventors: Tatsuo Kumazaki, Tokyo (JP); Satoru Murata, Tokyo (JP)

Correspondence Address: George A. Loud, Esquire;BACON & THOMAS, 625 Slaters Lane, Fourth Floor, Alexandria, VA, 22314-1176, US

(21) Appl. No.: 11/065,138

(22) Filed: Feb. 24, 2005

(30) Foreign Application Priority Data
Jul. 15, 2004 (JP) 2004-208970

(51) Int. Cl. A61M 29/00

(52) U.S. Cl. 604/101.04

(57) ABSTRACT

The present invention finds utility in the field of therapy of paraaortic lymph node tumors, and is directed to provisions of a drug perfusion method capable of preventing a drug delivered to the site of tumor tissue from leaking into the surrounding tissue regions, effectively exposing the lymph nodes to a drug of high concentration, and reducing or preventing adverse side effects which would otherwise be imposed on the patient; to a catheter-introducing sheath; and to an axygenated blood perfusion apparatus.

According to the invention, balloon catheters 32-1, 32-2, 32-3, 32-4, 32-5, 33, and 34 are employed to block the right renal artery 20, the left renal artery 21, the celiac artery 12, the superior mesenteric artery 19, the inferior mesenteric artery 24, the aorta 3, and the inferior vena cava 4, respectively, whereby a blood-vessel-blocked region is created. A non-branched sheath 40-3 is employed to deliver a drug into the blood-vessel-blocked region, and blood containing the administered drug is discharged through a non-branched sheath 40-4. During the period where the blood vessels remain blocked, oxygenated blood is supplied through the tip end of the balloon catheters 32-1, 32-2, 32-3, 32-4, 32-5, whereby organs are protected from loss of blood.

### BACKGROUND OF THE INVENTION

1. Field of the Invention

The present invention relates to a method of local drug delivery by way of perfusion; specifically, to delivery of a drug to paraaortic lymph node tumors by way of perfusion (hereinafter may be referred to as, for example, a “method of drug perfusion in paraaortic lymph node tumors”); to an apparatus for performing the method; and to a sheath used for performing the method. More particularly, the present invention relates to an effective method for delivering, by way of perfusion, a drug of high concentration to paraaortic lymph nodes, while preventing inflow of the drug to normal tissues, to thereby provide a chemical treatment against tumors; to an apparatus for performing the method; and to a sheath used for performing the method.

2. Description of the Related Art

The lymphatic system is constituted of lymph ducts spread throughout the body, and lymph nodes, which are nodules of tissue found along the lymph ducts. Lymph fluid containing lymphocytes flows through the lymph ducts. Lymphocytes produce antibodies for defending the living body against bacteria and viruses. Lymphocytes are differentiated and proliferated in the lymph nodes. The lymph nodes filter the lymph in order to remove foreign matter such as bacteria.

The lymph nodes are found systemically, but primarily in the armpits and the neck. Of such lymph nodes, those present around the abdominal aorta are called paraaortic lymph nodes. The aorta is the major artery of the cardiovascular system for pumping blood from the heart to the entire body. The paraaortic lymph nodes, which are located around the aorta, are connected to organs throughout the whole body through lymph ducts.

When a patient suffers stomach cancer or renal cancer as a result of the development of a malignant tumor primarily occurring in an organ other than the stomach or kidneys, the tumor may spread to the paraaortic lymph nodes through the lymph ducts around that organ. In such a case, as the conditions aggravate, tumor cells happen to pass through some lymph nodes, and ultimately the tumor may be spread systemically through the blood vessels and lymph ducts. Such tumors that have already been spread over the whole body are extremely difficult to cure, and the prognosis is generally very poor.

Hitherto, when a tumor is found in a paraaortic lymph node, therapy has followed either a surgical approach or a chemical approach.

In the surgical approach, lymph nodes are dissected (removed) by surgical operation and the tumor tissue is removed. Although this approach may promise a radical treatment, a great burden is imposed on the patient, since a large incision running from the abdomen to the chest is made, and not only lymph nodes but sometimes their surrounding tissue are also removed to a great extent. Moreover, there is a risk that the patient may be infected and develop, e.g., sepsis during or after the surgical operation.

Meanwhile, in the chemical approach, a drug such as an anticancer agent is administered to the tumor tissue, to thereby suppress differentiation of tumor cells and destroy the cells. In general, the drug is administered to the tumor tissue either via the oral route or by means of a catheter or a like apparatus, which is inserted into a blood vessel such as the jugular vein for delivering the drug to the tumor tissue through the liquid-feeding passage provided in the apparatus.

This approach neither leaves a long cut in the patient's skin nor removes tissue from the patient. Therefore, a smaller burden will be imposed on the patient. However, this approach cannot prevent the anticancer drug from migrating into another organ, thereby reducing the concentration of the drug to which the tumor tissue is exposed, and lowering the anti-tumor effect. Another possible disadvantage is that when the drug flows into unintended tissues; i.e., tissues other than the tumor tissue, normal cells will be injured, causing adverse side effects.

Conventionally, in the therapy of a pelvic tumor, perfusion with an anticancer agent has been performed in order to administer the agent effectively to the tumor tissue while preventing the above-mentioned adverse side effects. According to this method, two balloon catheters are employed such that one is indwelled in the aorta and the other in the vena cava, and the balloons are inflated for closure thereof to thereby block blood flow. Subsequently, tourniquets are placed on the limbs to stop blood circulation in the lower limbs. The balloon inflation regions of the balloons and the tourniquets mounted on the two limbs create a closed region isolated from blood circulation. The pelvic tumor tissue is contained in the thus-created closed region.

After blockage of the blood vessels, an anticancer agent is fed to the closed region through the artery site, and the bodily fluid is removed at a venous site. This method promises an enhanced anti-tumor effect and reduced side effects, because it enables targeted exposure of an anticancer agent of high concentration to only the tumor tissue, since the anticancer agent is administered after the arteriovenous blood flow has been cut, to thereby prevent the agent from being delivered to the whole body (see, for example, Patent Document 1).

Patent Document 1: Japanese Patent Application Laid-Open (kokai) No. 2003-102829

However, in the treatment of paraaortic lymph node tumors, perfusion in a manner as described above has not been performed, and demand still exists for development of effective treatment for such tumors.

### SUMMARY OF THE INVENTION

In view of the foregoing, an object of the present invention is to provide a drug perfusion method employable in the treatment of a tumor developed in paraaortic lymph nodes, the method ensuring effective exposure of the lymph nodes with a therapeutic agent of high concentration, while preventing the therapeutic agent fed to the tumor tissue from leaking into surrounding tissues, and reducing or preventing side effects attributable to the therapeutic agent; an apparatus for performing the drug perfusion method; and a sheath used for performing the drug perfusion method.

Another object of the present invention is to provide a catheter-introducing sheath employable in the treatment of a tumor developed in paraaortic lymph nodes, wherein the use of the sheath reduces the number of instruments which are percutaneously introduced into the body or a blood vessel of the patient and indwelled therein.

A further object of the present invention is to provide a catheter-introducing sheath capable of attaining smooth insertion of three catheters into the patient's body or blood vessels.

According to the invention described in claim 1, there is provided a drug perfusion method of delivering a drug by way of perfusion to a site of tumor tissue located in a paraaortic lymph node and recovering the drug, comprising:
a perfusion preparatory step including introducing a plurality of balloon catheters into a living body percutaneously so as to indwell the catheters in blood vessels which are branched from the aorta and are present around paraaortic lymph nodes, so that balloon inflation regions of the balloon catheters are positioned at predetermined sites of the blood vessels, and applying to predetermined positions of the lower limbs tourniquets having inflatable regions;
a closed-region creating step of creating a closed region of arteriovenous vessels by inflating the balloons of the balloon catheters and the inflatable regions of the tourniquets so as to cut blood flow to and from the closed region;
an oxygenated blood supply step of feeding oxygenated blood to organs to which blood supply has been cut as a result of the closed-region creating step having been performed;
a drug administration step of administering a drug solution containing a drug to the closed region; and
a bodily fluid recovering step of recovering from the closed region bodily fluid containing the drug.

According to the invention as described in claim 2, there is provided a drug perfusion method of delivering a drug by way of perfusion to a site of tumor tissue located in a paraaortic lymph node as recited in claim 1, wherein the balloons of the balloon catheters are indwelled so that the balloon inflation regions are positioned at predetermined sites in at least the right renal artery, the left renal artery, the celiac artery, the superior mesenteric artery, the inferior mesenteric artery, the aorta proximal to the heart with respect to the branching portion of the celiac artery, and the inferior vena cava.

Thus, the perfusion method as recited in claim 1 or claim 2 includes closing major branching blood vessels of the aorta to thereby create a blood vessel closure region around the tumor tissue, and subsequent administering of a drug to the closed region. Since the drug administered to the closed region remains to stay within the region, the concentration of the drug within the region can be maintained high, and therefore, lymph node tumors can be effectively exposed to the drug of high concentration.

Also, since the perfusion method as recited in claim 1 or claim 2 prevents the drug from coming into contact with normal tissues that are present outside the closed region, side effects of the drug can be reduced or prevented.

Moreover, according to the perfusion method as recited in claim 1 or claim 2, oxygenated blood is fed to organs to which blood supply has been cut. Therefore, ischemia of the organs which would otherwise be caused due to stopped blood flow is prevented, to thereby reduce or prevent the risk of injuring the organs.

According to the invention described in claim 3, oxygenated blood is fed to organs through a balloon catheter indwelled in the right renal artery, a balloon catheter indwelled in the left renal artery, and at least one balloon catheter selected from among the three balloon catheters indwelled at predetermined positions in the celiac artery, the superior mesenteric artery, and the inferior mesenteric artery.

Thus, according to the perfusion method as recited in claim 3, in the case of feeding oxygenated blood to the celiac artery, the superior mesenteric artery, and the inferior mesenteric artery, feeding of oxygenated blood through only one of these three arteries can transport oxygenated blood to all the organs that communicate with these three arteries. Therefore, cumbersome manipulation of feeding blood to all three of the arteries simultaneously is no longer necessary, and organs can be protected with a simplified blood feeding procedure.

According to the invention described in claim 4, a drug is administered through a balloon catheter which is indwelled in the aorta at a predetermined position thereof, the position being on the heart side with respect to the branching portion of the celiac artery.

Thus, according to the perfusion method as recited in claim 4, a single balloon catheter indwelled in the aorta at a predetermined position on the heart side of the branching portion is used to inflate the balloon and also to administer the drug. Therefore, the number of instruments to be inserted into the patient's body can be reduced, to thereby reduce the burden imposed on the patient.

According to the invention described in claim 5, there is provided a catheter-introducing sheath for introducing into a living body a catheter which is used for performing the perfusion method of delivering, by way of perfusion, a drug to a paraaortic lymph node tumor, comprising:
a first passage for allowing passage of a first catheter therethrough, a second passage branching from the first passage and allowing passage of a second catheter therethrough, and a third passage branching from the first passage and allowing passage of a third catheter therethrough.

Thus, the catheter-introducing sheath as recited in claim 5 has a structure which allows insertion of three catheters simultaneously. Therefore, use of the catheter-introducing sheath of the present invention can reduce the number of instruments to be percutaneously inserted into the patient's body, to thereby reduce the burden imposed on the patient.

According to the invention described in claim 6, there is provided a catheter-introducing sheath for introducing into a living body a catheter which is used for performing the perfusion method of delivering, by way of perfusion, a drug to a paraaortic lymph node tumor, comprising:
a tubular body having a first passage for allowing passage of a catheter therethrough,
a first branched connector which is connected to an end of the tubular body and which has a second passage and a third passage, the second passage directly communicating, via a first end of the first branched connector, with the first passage and the third passage branching from the second passage, and
a second branched connector which is connected to a second end of the first branched connector, the second end being opposite the first end, and which has a fourth passage and a fifth passage, the fourth passage directly communicating with the second passage and the fifth passage branching from the fourth passage.

According to the invention described in claim 7, the first and the second branched connectors in the catheter-introducing sheath as recited in claim 6 are made of the same material.

According to the invention described in claim 8, the axis running through the first, second, and the fourth passages, the axis running through the third passage, and the axis running through the fifth passage do not lie within a single plane.

Thus, since the catheter-introducing sheath according to any of claim 6, 7 or 8 is constructed by connecting existing two-way connectors, design modification for fabricating a three-way connector is no longer necessary.

Also, in the catheter-introducing sheath according to any of claim 6, 7 or 8, the two branching portions, one in the first branched connector and the other in the second branched connector, are located apart from each other and at offset position. Therefore, when a balloon catheter is inserted into one connector through its branch tube, the front end of the balloon catheter is prevented from being caught at the branching portion of the other connector, ensuring smooth insertion of the balloon catheter.

Moreover, the catheter-introducing sheath according to any of claim 6, 7 or 8 is constructed such that the axis of the branching tube of the first branched connector, the axis of the branching tube of the second branched connector, and the axis of the first passage do not lie within a single plane. Therefore, when a balloon catheter is inserted through the branching tube of the second branched connector, the incident of catheter entering the branching tube of the first branched connector and blocking the branching portion thereof will not occur, whereby a smooth insertion of the balloon catheter can be attained.

According to the invention described in claim 9, there is provided a catheter introducer for introducing into a living body a catheter-inserting sheath which is used for performing the perfusion method of delivering, by way of perfusion, a drug to a paraaortic lymph node tumor, comprising:
a catheter-inserting sheath, and
a dilator;
   wherein the catheter-inserting sheath comprises:
a first passage for allowing passage of the first catheter therethrough,
a second passage branching from the first passage and allowing the second catheter to pass therethrough, and
a third passage branching from the first passage and allowing the third catheter to pass therethrough.

According to the invention described in claim 10, there is provided a catheter introducer for introducing into a living body a catheter-inserting sheath which is used for performing the perfusion method of delivering, by way of perfusion, a drug to a paraaortic lymph node tumor, comprising:
a catheter-inserting sheath, and
a dilator;
   wherein the catheter-inserting sheath comprises:
a tubular body having a first passage for allowing passage of a catheter therethrough,
a first branched connector which is connected to an end of the tubular body and which has a second passage and a third passage, the second passage directly communicating, via a first end of the first branched connector, with the first passage and the third passage branching from the second passage, and
a second branched connector which is connected to a second end of the first branched connector, the second end being opposite the first end, and which has a fourth passage and a fifth passage, the fourth passage directly communicating with the second passage and the fifth passage branching from the fourth passage.

According to the invention described in claim 11, the dilator of the catheter introducer as recited in claim 9 is inserted into the above-mentioned first passage. Also, according to the invention described in claim 12, the dilator of the catheter introducer as recited in claim 10 is inserted into a passage formed by the first, second, and the third passage.

As described above, the catheter introducer according to any of claim 9, 10, or 11 has a three-way sheath-introducer. Therefore, the number of instruments to be percutaneously inserted into the patient's body can be reduced, to thereby reduce the burden of the patient.

In the catheter introducer of the present invention, a dilator is inserted through straight-channel-forming passages among a plurality of passages. Therefore, insertion of the dilator into the sheath and removal therefrom can be performed smoothly.

According to the invention described in claim 13, there is provided an oxygenated blood perfusion apparatus equipped with fluid discharge means for discharging bodily fluid out of a living body, bodily-fluid-oxygenating means for oxygenating the discharged bodily fluid, and feeding means for feeding oxygenated blood into the living body through a plurality of balloon catheters indwelled in the living body, wherein any one to three of said plurality of balloon catheters are respectively inserted into the living body through their corresponding catheter-inserting sheaths, each sheath comprising:
a first passage for allowing passage of the first catheter therethrough,
a second passage branching from the first passage and allowing the second catheter to pass therethrough, and
a third passage branching from the first passage and allowing the third catheter to pass therethrough.

According to the invention described in claim 14, there is provided an oxygenated blood perfusion apparatus equipped with fluid discharge means for discharging bodily fluid out of a living body, bodily-fluid-oxygenating means for oxygenating the discharged bodily fluid, and feeding means for feeding oxygenated blood into the living body through a plurality of balloon catheters indwelled in the living body, wherein one to three of said plurality of balloon catheters are respectively inserted into the living body through their corresponding catheter-inserting sheaths, each sheath comprising:
a tubular body having a first passage for allowing passage of a catheter therethrough,
a first branched connector which is connected to an end of the tubular body and which has a second passage and a third passage, the second passage directly communicating, via a first end of the first branched connector, with the first passage and the third passage branching from the second passage, and
a second branched connector which is connected to a second end of the first branched connector, the second end being opposite the first end, and which has a fourth passage and a fifth passage, the fourth passage directly communicating with the second passage and the fifth passage branching from the fourth passage.

Thus, the oxygenated blood perfusion apparatus as recited in claim 13 or 14 includes a plurality of balloon catheters connected to the apparatus and inserted into a living body via catheter-inserting sheaths, each having three passageways. Thus, the number of instruments which are percutaneously introduced into the body or a blood vessel of the patient and indwelled therein can be reduced.

As described above, the present invention attains effective exposure of lymph nodes to a drug of high concentration. Also, the invention reduces or prevents adverse side effects attributable to the drug. Moreover, the invention reduces or prevents injury of organs which would otherwise occur due to stopped blood flow. Furthermore, the invention reduces or prevents organs that communicate with the right renal artery, the left renal artery, the celiac artery, the superior mesenteric artery and the inferior mesenteric artery from being injured due to loss of blood, etc.

### BRIEF DESCRIPTION OF THE DRAWINGS

Various other objects, features, and many of the attendant advantages of the present invention will be readily appreciated as the same becomes better understood with reference to the following detailed description of the preferred embodiments when considered in connection with accompanying drawings, in which:

FIG. 1 is an explanatory view schematically showing the blood vessels and branches thereof and locations of catheters inserted according to an embodiment of the present invention;

FIG. 2 is an explanatory enlarged view of the area surrounded by a broken line in FIG. 1;

FIG. 3 is a sketch of a non-branched introducer according to an embodiment of the present invention;

FIG. 4 is an elevational sectional view of a non-branched sheath according to an embodiment of the present invention;

FIG. 5 is a sketch of a three-way introducer according to an embodiment of the present invention;

FIG. 6 is an elevational sectional view of a three-way sheath according to an embodiment of the present invention;

FIG. 7 is an elevational sectional view of a balloon catheter according to an embodiment of the present invention;

FIG. 8 is an elevational sectional view of another balloon catheter according to an embodiment of the present invention;

FIG. 9 is an explanatory view showing the configurations of perfusion circuits A and B according to an embodiment of the present invention;

FIG. 10 is an explanatory view showing a settings display panel of a perfusion apparatus employed in the perfusion circuit A according to an embodiment of the present invention;

FIG. 11 is an explanatory view showing a settings display panel of a perfusion apparatus employed in the perfusion circuit B according to an embodiment of the present invention;

FIG. 12 is a flow chart of the perfusion process according to an embodiment of the present invention; and

FIG. 13 is a graph representing experimental data obtained in an embodiment of the present invention.

### DETAILED DESCRIPTION OF THE INVENTION

Some embodiments of the present invention will next be described with reference to the drawings. The elements, configurations and the like described hereinbelow should not be construed as limiting the invention thereto. As will be appreciated, various modifications may be made without departing from the scope of the invention. For example, a non-branched sheath 40-1, which is inserted into the right axillary artery in an embodiment, may be inserted into the brachial artery.

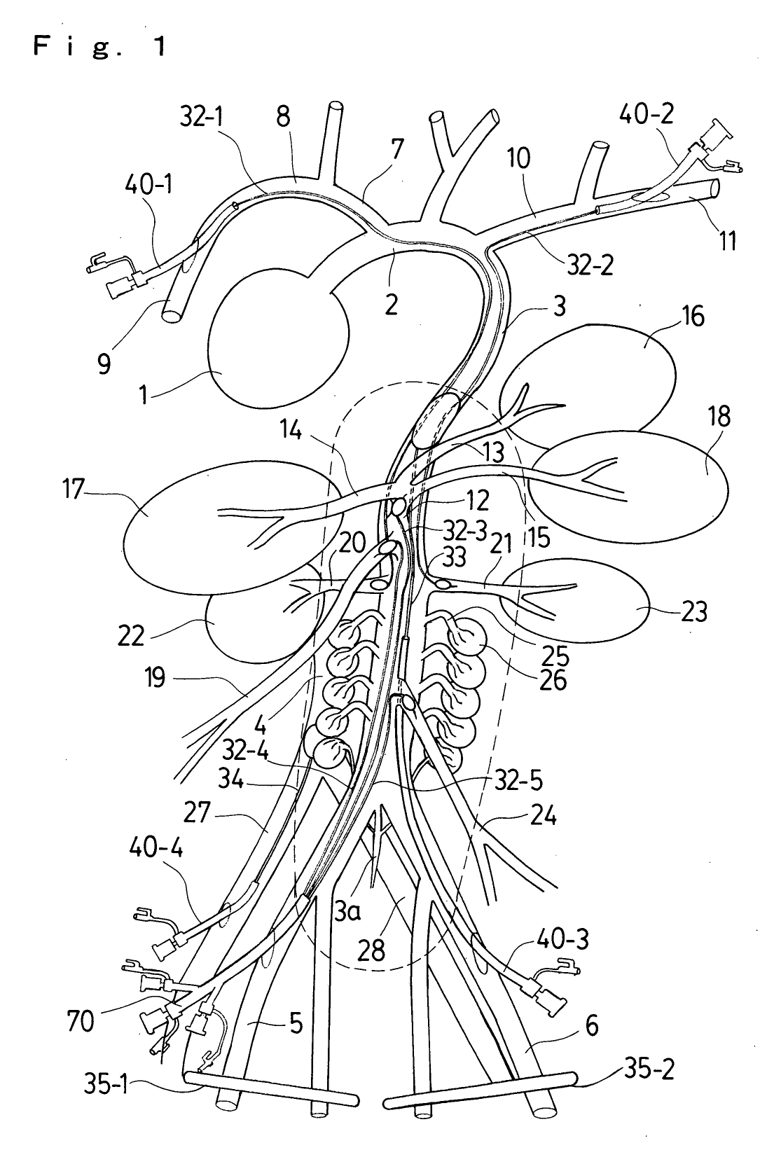

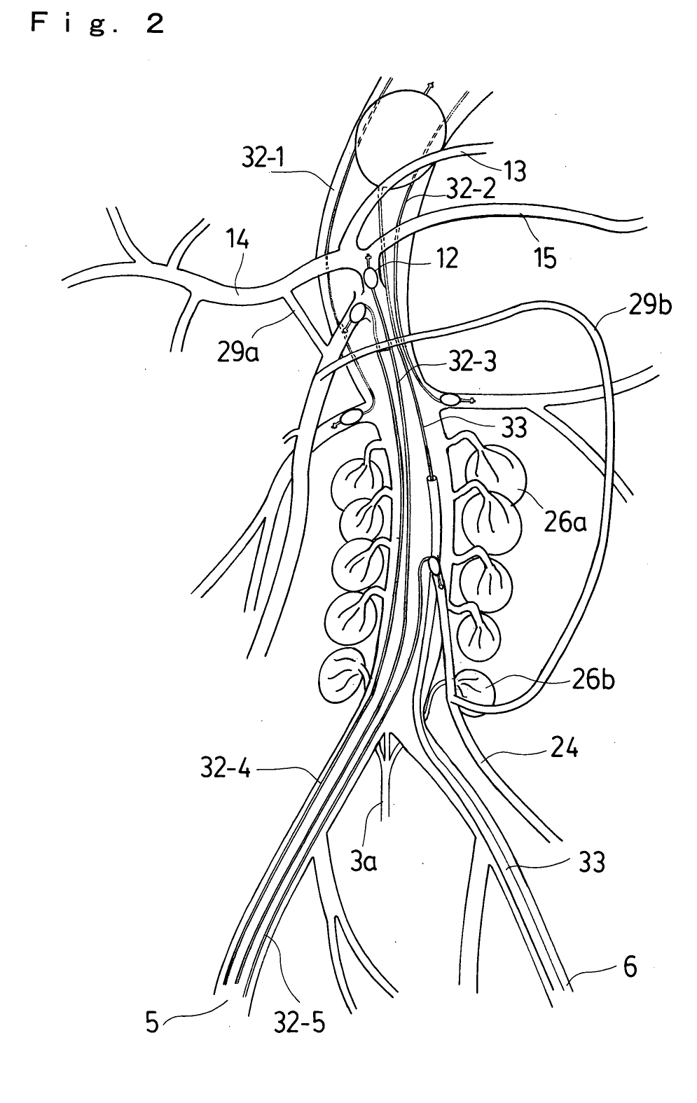

FIGS. 1 and 2 show an embodiment of the drug perfusion method according to the present invention as applied to a case in which a malignant pelvic tumor spreads to the paraaortic lymph nodes. FIG. 1 is an illustration of the embodiment of the present invention showing the branches of blood vessels and the insertion positions of catheters. FIG. 2 shows an enlarged illustration showing the area enclosed by the broken line in FIG. 1. In FIGS. 1 and 2, balloon catheters hidden by blood vessels or equipment are represented by broken lines in order to facilitate understanding of the invention.

Firstly, the main organs and the branches of blood vessels around the aorta will be described. A blood vessel originating from the left ventricle of the heart 1 extends to the aorta 3 via the aortic arch 2, and the aorta 3 is branched into the common right and left iliac arteries at the pelvis. The common right iliac artery communicates with the right femoral artery 5, and the left common iliac artery communicates with the left femoral artery 6.

The aortic arch 2 has a branch to the brachiocephalic artery 7, and the brachiocephalic artery 7 is branched into the right subclavian artery 8 and the right common carotid artery. The right subclavian artery 8 runs on the lateral side of the first rib, where it changes its name to right axillary artery 9. The aortic arch 2 has another branch to the left subclavian artery 10, located downstream of the branch to the brachiocephalic artery 7. The left subclavian artery 10 runs on the lateral side of the first rib, where it changes its name to left axillary artery 11.

The aorta 3 has a branch en route to the celiac artery 12, and the celiac artery 12 is branched into the splenic artery 15, the gastric coronary artery 13, and the common hepatic artery 14. The splenic artery 15, the gastric artery 13, and the hepatic artery 14 communicate with the spleen 18, the stomach 16, and the liver 17, respectively.

Downstream of the branch to the celiac artery 12, the aorta 3 has another branch to the superior mesenteric artery 19, which communicates with the small intestine (not shown). Downstream of the branch to the superior mesenteric artery 19, the aorta 3 further has branches to the right renal artery 20 and left renal artery 21, which communicate with the right kidney 22 and the left kidney 23, respectively. Downstream of the branches to the right renal artery 20 and left renal artery 21, the aorta 3 has a branch to the inferior mesenteric artery 24, which communicates with the hindgut (not shown).

The first collateral pathway 29a extends between the common hepatic artery 14 and the superior mesenteric artery 19, and the second collateral pathway 29b extends between the superior mesenteric artery 19 and the inferior mesenteric artery 24.

Downstream of the branches to the right renal artery 20 and left renal artery 21, the aorta 3 has 4 branches to the first to fourth lumbar arteries on each of the left and right sides. The first to fourth lumbar arteries communicate with the first to fourth paraaortic lymph nodes, respectively. The middle sacral artery 3a is branched from the branch point of the right femoral artery 5 and the left femoral artery 6. The middle sacral artery 3a also has branches on the left and right sides to the fifth lumbar arteries. The left and right fifth lumbar arteries communicate with the respective fifth paraaortic lymph nodes. In FIG. 1, the first to fifth lumbar arteries are represented by reference numeral 25, and the first to fifth paraaortic lymph nodes are represented by reference numeral 26.

An artery branches into capillary vessels in each organ and tissue to supply nutrients and oxygen to the organ and the cells. After supplying nutrients and oxygen, blood receives waste products and carbon dioxide and returns to capillary vessels. The capillary vessels then merge to form a vein.

The right femoral vein 27 in the right lower limb and the left femoral vein 28 in the left lower limb merge at the abdomen to form the inferior vena cava 4. The inferior vena cava 4 communicates with the right atrium of the heart downstream of the blood flow.

The following description is based on the assumption that a patient has a tumor in a paraaortic lymph node 26. Examples of the primary focus of a tumor in the paraaortic lymph node include malignant pelvic tumors such as bladder cancer and uterus carcinoma. Bladder cancer is caused by canceration of the transitional epithelium, which covers the surface of the bladder and has high stretchability. Although bladder cancer tends to occur inside the bladder, similar lesions may also be found in the ureters and the renal pelvises, which are located upstream of urine flow. Uterus carcinoma includes uterus sarcoma, cervical carcinoma, and endometrial carcinoma, among others. When malignant pelvic tumors advance, tumor cells may spread to the paraaortic lymph nodes through the lymph ducts or the blood vessels around the bladder or the uterus.

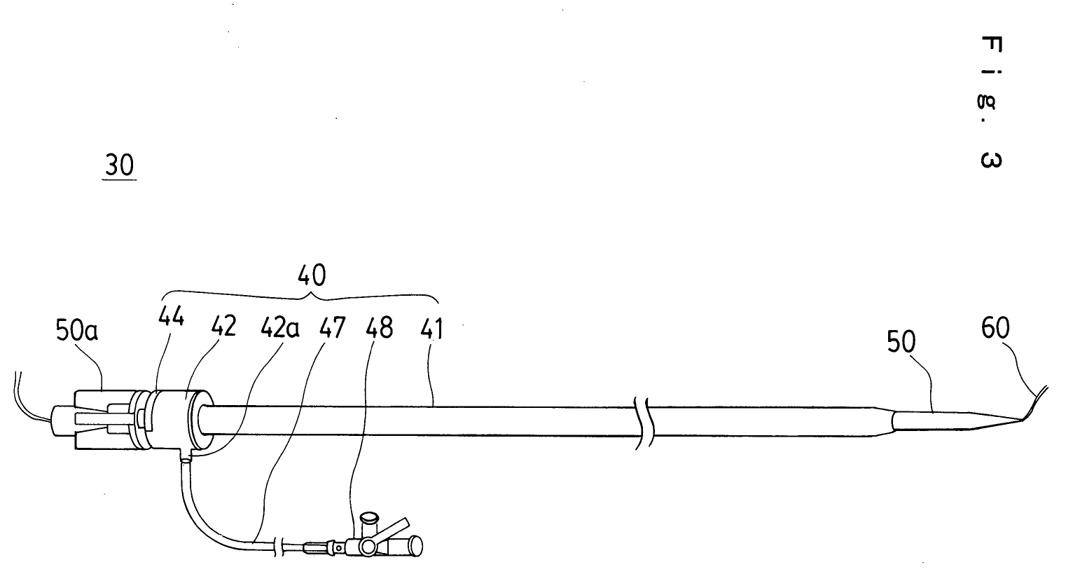

Sheath introducers, sheaths, and catheters according to the present invention will next be described with reference to FIGS. 3 to 8.

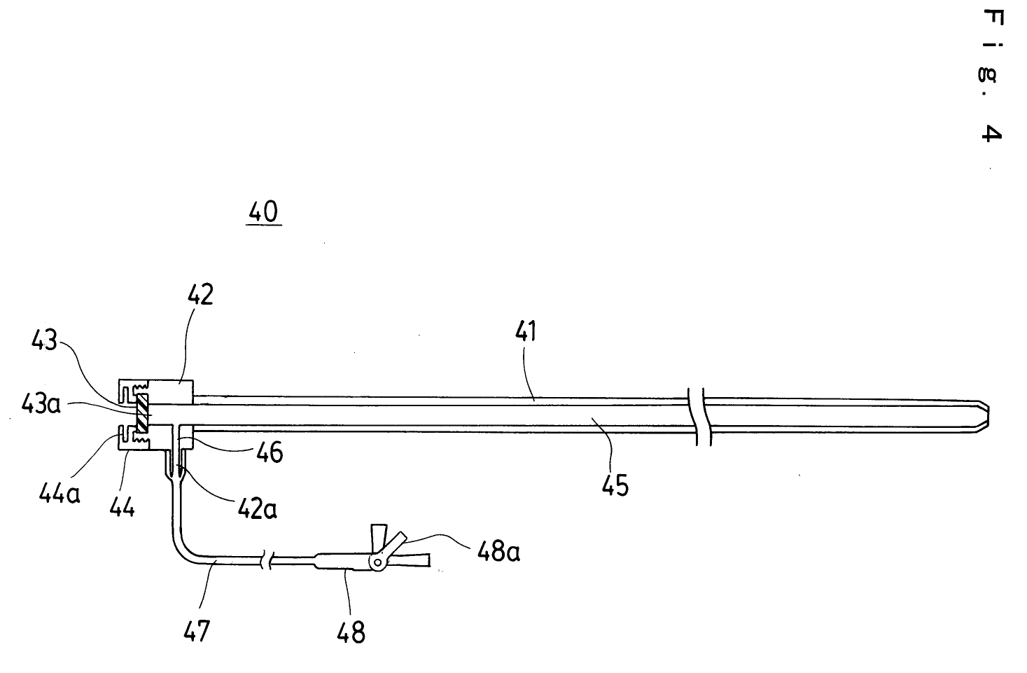

FIGS. 3 and 4 show the structure of a non-branched introducer 30 according to the present invention. FIG. 3 is a sketch of the non-branched introducer 30 according to an embodiment of the present invention. FIG. 4 is an elevational sectional view of a non-branched sheath 40 according to an embodiment of the present invention.

The non-branched introducer 30 is used as a guide for a balloon catheter when the catheter is percutaneously inserted into a blood vessel. The non-branched introducer 30 has, as primary components, the non-branched sheath 40, a dilator 50, and a guide wire 60.

The non-branched sheath 40 has a tubular body 41 and a rear end portion 42 as major components. The tubular body 41 and the rear end portion 42 are connected together through a known technique such as application of an adhesive or melt adhesion.

The tubular body 41 is a flexible or rigid tubular member made of polytetrafluoroethylene (PTFE). The tubular body 41 has a passage provided therein, through which the opposite ends of the tubular body 41 communicate with each other. One end portion of the tubular body 41 has a diameter decreasing along the direction of insertion of the sheath. Thus, the tubular body 41 and the dilator 50 form no step therebetween, allowing smooth insertion of the non-branched introducer 30 into the blood vessel.

The rear end portion 42 is a flexible or rigid cylindrical member made of polytetrafluoroethylene (PTFE). The rear end portion 42 is attached to the rear end of the tubular body 41. The rear end portion 42 has a passage provided therein which communicates with the passage of the tubular body 41. In FIG. 4, the passage of the tubular body 41 and the passage of the rear end portion 42 together form a passage indicated by reference number 45.

The rear end portion 42 has a hemostatic valve 43 provided therein made of an elastic material such as a synthetic rubber or a thermoplastic elastomer. The circumferential perimeter of the hemostatic valve 43 is held by sandwiching between the rear end portion 42 and a cap member 44, to thereby fix the hemostatic valve 43 in the rear end portion 42. The cap member 44 has a thread 44a, with which the cap member 44 is engaged with a thread portion (not shown) provided in the rear end portion 50a of the dilator.

The hemostatic valve 43 is provided with a small hole 43a at a center portion thereof, through which opposite sides of the hemostatic valve 43 communicate with each other. A dilator or a balloon catheter is inserted into the passage 45 through the small hole 43a.

The rear end portion 42 has a projection 42a extending from a circumferential portion thereof. The projection 42a has a side port 46 provided therein, through which the passage 45 communicates with the tip of the projection 42a. A flexible tube 47 is connected to the projection 42a, and a three-way cock 48 is attached to the other end of the flexible tube 47.

The three-way cock 48 has a plurality of ports. The three-way cock 48 has a selector valve which functions to switch communication between the tube 47 and one of these ports. A liquid-feeding pump or a liquid-removing pump can be connected to the ports of the three-way cock 48. Through use of such a liquid-feeding pump, a drug solution or other liquids (hereinafter simply referred to as “a drug solution”) is fed into a living body sequentially via the three-way cock 48, the tube 47, the side port 46, and the passage 45. Through use of such a liquid-removing pump, bodily fluid is removed from the living body sequentially via the passage 45, the tube 47, and the three-way valve 48.

The dilator 50 is a flexible stick-shaped member made of polytetrafluoroethylene (PTFE). The dilator 50 is used for preventing the tubular body 41 from kinking on the occasion of insertion of the non-branched introducer 30 into the blood vessel. The dilator 50 has a tubular passage (not shown) provided therein for allowing the tip and rear ends of the dilator to communicate with each other. The guide wire 60 can be inserted into the tubular passage.

The dilator 50 is inserted into the passage 45 of the non-branched sheath 40 through the small hole 43a of the hemostatic valve 43. The tip portion of the dilator 50 projects over the tip portion of the non-branched sheath 40. The rear portion of the dilator 50 is adapted to screw into the thread 44a of the cap member 44. This structure prevents relative movement between the dilator 50 and the non-branched introducer 30 which may otherwise occur when the introducer 30 is inserted into the blood vessel.

The guide wire 60 is a member for leading the tip portion of the dilator 50 to a target site. The guide wire 60 is a filament-shaped member made of a material such as stainless steel or a nickel-titanium alloy. The guide wire 60 is inserted into the tubular passage (not shown) of the dilator 50. The tip portion of the guide wire 60 projects over the tip portion of the dilator 50, and the rear end portion of the guide wire 60 projects over the rear end portion of the dilator 50.

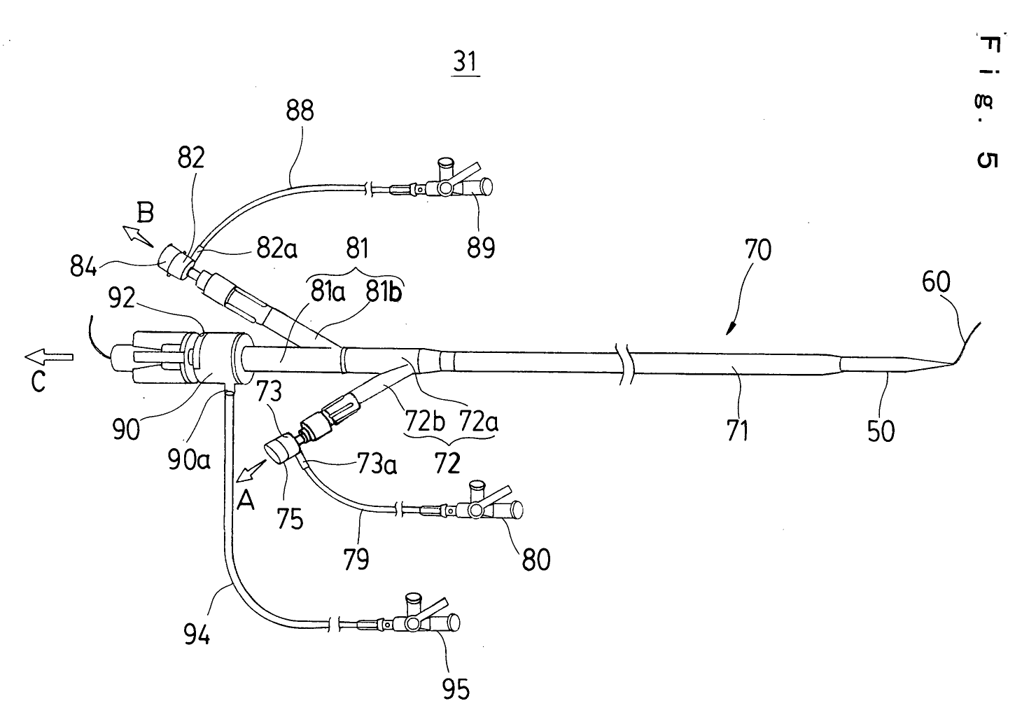

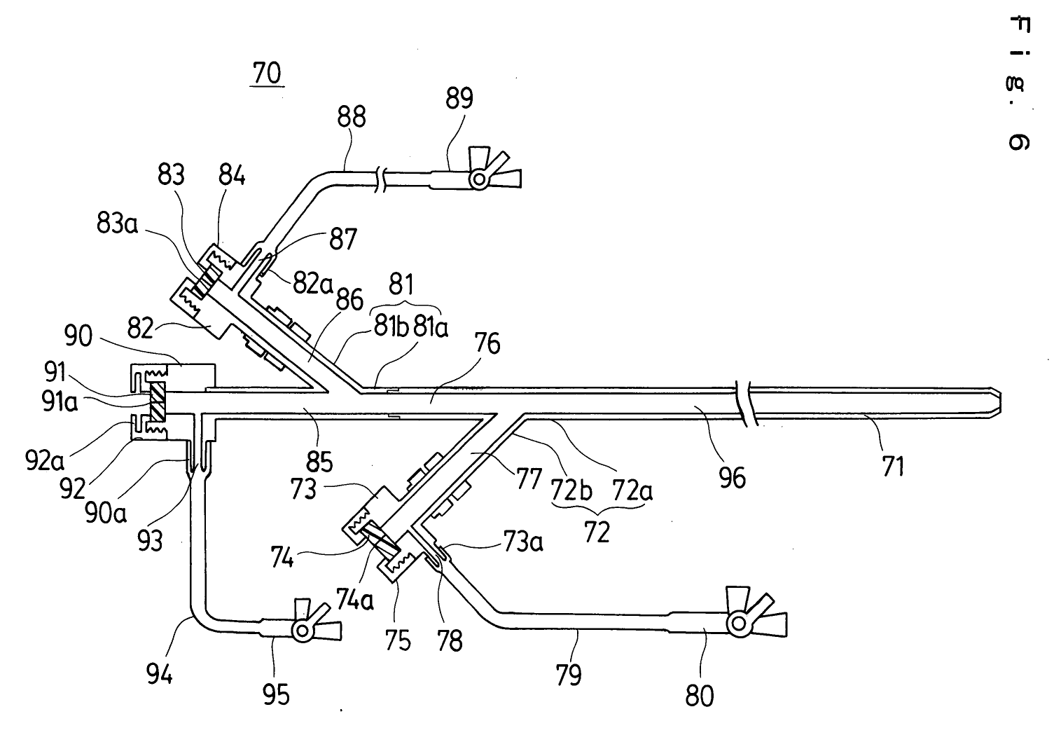

FIGS. 5 and 6 show the structure of a three-way introducer 31. FIG. 5 is a sketch of the three-way introducer 31 according to an embodiment of the present invention. FIG. 6 is an elevational sectional view of a three-way sheath 70 according to an embodiment of the present invention.

The three-way introducer 31 is used as a guide for a balloon catheter when the catheter is percutaneously inserted into a blood vessel. The three-way introducer 31 has, as primary components, a three-way sheath 70, a dilator 50, and a guide wire 60. The dilator 50 and the guide wire 60 may be the same as those employed in the above-mentioned non-branched introducer 30.

The three-way sheath 70 has a tubular body 71, a first branched connector 72, a second branched connector 81, a first rear portion 73, a second rear portion 82, and a third rear portion 90.

The tubular body 71 is a flexible or rigid tubular member made of PTFE. The tubular body 71 has a length of 300 mm and a diameter of 5 mm, and has a passage 96 provided therein, through which the opposite ends of the tubular body 71 communicate with each other. One end portion of the tubular body 71 has a diameter decreasing along the direction of insertion of the sheath. Thus, the tubular body 71 and the dilator 50 form no step therebetween, allowing smooth insertion of the three-way introducer 31 into a blood vessel.

The first branched connector 72 is a flexible or rigid member made of a material such as PTFE. The first branched connector 72 has a cylindrical main tube 72a, and a branch tube 72b branched from the main tube 72a.

The main tube 72a is in the form of a hollow cylinder having a length of 25 mm and a diameter of 6 mm, and has a passage 76, through which the opposite ends of the main tube 72a communicate with each other. One end of the main tube 72a is connected to one end of the tubular body 71 through a known technique such as adhesion. The passage 76 of the main tube 72a is connected directly to the passage 96 of the tubular body 71. The other end of the main tube 72a is connected to a main tube 81a of the second branched connector 81. The branch tube 72b is branched from the main tube 72a at a portion about 8 mm from the connection end of the main tube 72a to the tubular body 71, such that the inner angle formed by the main tube 72a and the branch tube 72b is about 30°.

The branch tube 72b is in the form of a hollow cylinder having a length of 15 mm and a diameter of 6 mm, and has a passage 77, through which the rear end of the branch tube 72b communicates with the passage 76. The open end of the branch tube 72b is connected to a rear end portion 73. The rear end portion 73 has a hemostatic valve 74 provided therein made of an elastic material such as a synthetic rubber or a thermoplastic elastomer. The circumferential perimeter of the hemostatic valve 74 is held by sandwiching between the rear end portion 73 and a cap member 75, to thereby fix the hemostatic valve 74 in the rear end portion 73. The hemostatic valve 74 is provided with a small hole 74a at a center portion of the valve 74. A balloon catheter is inserted through the small hole 74a sequentially into the passages 77, 76, and 96.

The rear end portion 73 has a projection 73a extending from a circumferential portion thereof. The projection 73a has a side port 78 provided therein, through which the passage 77 communicates with the tip of the projection 73a.

The second branched connector 81 is a flexible or rigid member made of PTFE. The second branched connector 81 is identical with the first branched connector 72 mentioned above, and has a main tube 81a, and a branch tube 81b branched from the main tube 81a.

The main tube 81a is in the form of a hollow cylinder having a length of 25 mm and a diameter of 6 mm, and has a passage 85, through which the opposite ends of the main tube 81a communicate with each other. One end of the main tube 81a is connected to one end of the main tube 72a of the first branched connector 72 through a known technique such as adhesion. The passage 85 of the main tube 81a is connected directly to the passage 76 of the first branched connector 72. The other end of the main tube 81a is connected to the third rear portion 90. The branch tube 81b is branched from the main tube 81a at the point about 8 mm from the end of the main tube 81a connected to the first branched connector 72, such that the inside angle formed by the main tube 81a and the branch tube 81b is about 30°.

The branch tube 81b is in the form of a hollow cylinder having a length of 15 mm and a diameter of 6 mm, and has a passage 86, through which the rear end of the branch tube 81b communicates with the passage 85. The open end of the branch tube 81b is connected to the second rear portion 82. The second rear portion 82 has a hemostatic valve 83 provided therein made of an elastic material such as a synthetic rubber or a thermoplastic elastomer. The circumferential perimeter of the hemostatic valve 83 is held by sandwiching between the second rear portion 82 and a cap member 84, to thereby fix the hemostatic valve 83 in the second rear portion 82. The hemostatic valve 83 is provided with a small hole 83a at a center portion thereof, through which opposite sides of the hemostatic valve 83 communicate with each other. A balloon catheter is inserted through the small hole 83a sequentially into the passages 86, 85, 76, and 96.

The second rear portion 82 has a projection 82a extending from a circumferential portion thereof, through which opposite sides of the hemostatic valve 83 communicate with each other. The projection 82a has a side port 87 provided therein, through which the passage 86 communicates with the tip of the projection 82a.

The third rear portion 90 is connected to the other end of the main tube 81a. The third rear portion 90 has a hemostatic valve 91 provided therein made of an elastic material such as a synthetic rubber or a thermoplastic elastomer. The circumferential perimeter of the hemostatic valve 91 is held by sandwiching between the rear end portion 90 and a cap member 92, to thereby fix the hemostatic valve 91 in the rear end portion 90. The cap member 92 has a thread 92a, with which the cap member 92 is engaged with a thread portion (not shown) provided in the rear end portion of the dilator 50a. The hemostatic valve 91 is provided with a small hole 91a at a center portion thereof, through which opposite sides of the hemostatic valve 83 communicate with each other. A dilator or a balloon catheter is inserted through the small hole 91a sequentially into the passages 85 and 96.

The third rear portion 90 has a projection 90a extending from a circumferential portion thereof. The projection 90a has a side port 93 provided therein, through which the passage 85 communicates with the tip of the projection 90a.

Flexible tubes 79, 88, and 94 are connected to the projections 73a, 82a, and 90a, respectively. Three-way cocks 80, 89, and 95 are attached to the other ends of the flexible tubes 79, 88, and 94, respectively. Each of the three-way cocks has a plurality of ports. Through the tubes and the side ports, for example, introduction of saline into the passage 96 or removal of bodily fluid can be performed.

The second branched connector 81 is preferably identical with the first branched connector 72. As these connectors, connectors generally used for two-way catheter sheaths may be employed. That is, according to the present invention, typical two-way connectors can effectively be utilized to produce three-way connectors. Thus, three-way sheaths can be produced at low cost without newly designing three-way connectors. In addition, through use of three or more two-way connectors, four-way sheaths or higher-way sheaths can be produced.

Moreover, since the first branched connector 72 and the second branched connector 81 are connected with each other as described above, the portion at which the passage 77 is branched from the passage 76 and the portion at which the passage 86 is branched from the passage 85 are spaced from each other by a predetermined distance. This structure prevents the tip of a balloon catheter inserted via one branch tube from being caught at the other branch portion formed by the main tube and the other branch tube. Thus, according to the present invention, smooth insertion of a balloon catheter can be performed.

Further, as shown in FIG. 5, the axis A of the branch tube of the first branched connector 72, the axis B of the branch tube of the second branched connector 81, and the axis C of the passage 96 are not coplanar. This structure successfully prevents a balloon catheter inserted via the branch tube of the second branched connector 81 from unfavorably entering into and plugging the branch tube of the first branched connector 72, which may otherwise occur.

Next will be described balloon catheters employed in the present invention. In one embodiment of the present invention, three types of balloon catheters are used.

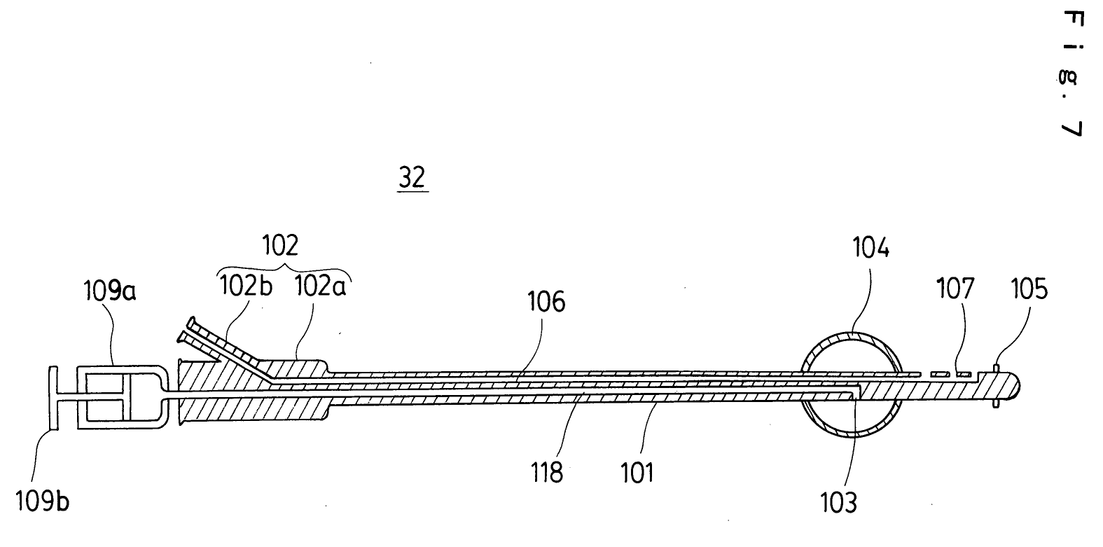

FIG. 7 is an elevational sectional view of a balloon catheter 32 according to such an embodiment of the present invention. In the present embodiment, the balloon catheter 32 is used as a first balloon catheter. The balloon catheter 32 has, as primary components, a basal portion 101, a rear end portion 102, a branch tube 102b, a balloon 104, a front coil marker 105, a tubular passage 108, a lumen 106, a syringe 109a, and a plunger 109b.

The basal portion 101 is a flexible member made of a resin such as polyethylene, polyethylene terephthalate, nylon, or polyester. The basal portion 101 has the tubular passage 108 provided therein. Through the tubular passage 108, the rear end of the rear end portion 102 communicates with a balloon-inflating opening 103 provided at a center portion of the balloon 104. The rear end portion 102 is connected to the syringe 109a enclosing a liquid (not shown) such as saline. When an operator applies pressure on the plunger 109b of the syringe 109a, the liquid flows through the tubular passage 108 and the balloon-inflating opening 103 into the inner space of the balloon 104.

The balloon 104 is a flexible member made of a resin such as polyethylene, polyethylene terephthalate, nylon, or polyester. The material forming the balloon 104 is softer than that of the basal portion 101. In addition, the wall forming the balloon 104 is thinner than that of the basal portion 101. Thus, when the liquid flows into the inner space of the balloon 104, the balloon 104 inflates. The balloon 104 is inflated until the balloon 104 comes into close contact with the inside wall of the blood vessel, to thereby block the blood flow in the blood vessel.

The basal portion 101 has a lumen 106 provided therein. Through the lumen 106, the rear end of the branch tube 102b communicates with side openings 107. The side openings 107 are provided in the basal portion 101 between the balloon 104 and the tip end of the basal portion 101. Therefore, even after the balloon 104 is inflated to plug the blood vessel, a drug solution can be fed through the lumen 106 to the region where the tip end of the basal portion exists and which is blocked by the balloon.

The rear end of the branch tube 102b may be connected to, for example, a syringe containing a drug solution or a liquid-feeding pump (not shown). Through use of such a syringe or a pump, a drug or other substances can be fed through the lumen 106 and the side openings 107 to the region where the tip end of the basal portion exists and which is defined by the blood vessel and the balloon which blocks blood flow.

The front coil marker 105 is made of a radiopaque material such as titanium. The front coil marker 105 allows an operator to specify the position of the tip of the balloon catheter 32 when carrying out an intervention under radioscopy. Provision of the front coil marker 105 enables the operator to selectively advance the balloon 104 toward the target site through confirmation of the position of the front coil marker 105 during angiography under X-ray fluoroscopy.

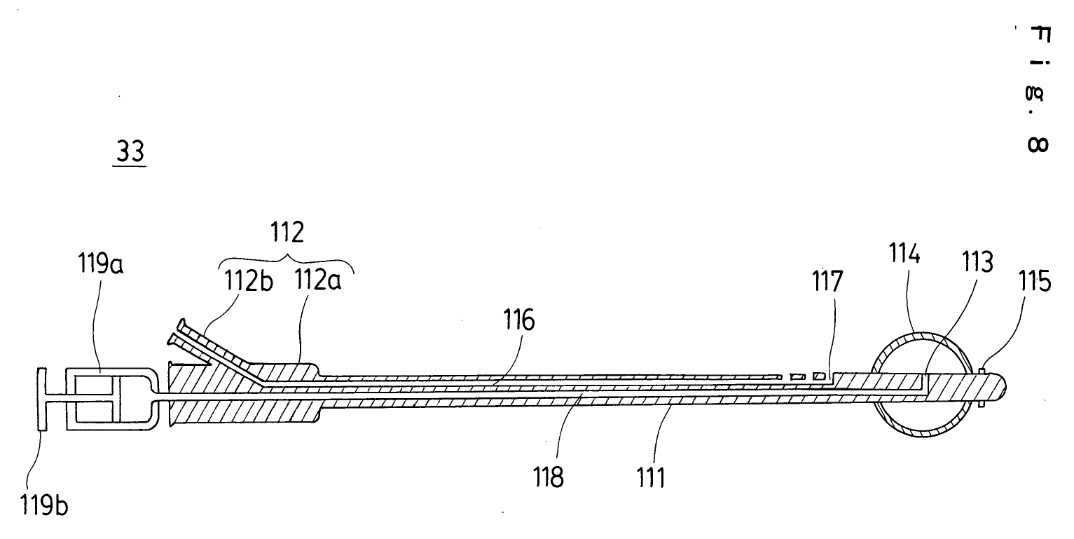

FIG. 8 is an elevational sectional view of another balloon catheter 33 according to an embodiment of the present invention. The balloon catheter 33 is used as a second balloon catheter in the embodiment according to the present invention. The balloon catheter 33 has a basal portion 111, a rear end portion 112, a branch tube 112b, an inflatable balloon 114, a front coil marker 115, a tubular passage 118, a lumen 116, a syringe 119a, and a plunger 119b.

The basal portion 111, the rear end portion 112, the branch tube 112b, the balloon 114, the front coil marker 115, the tubular passage 118, the syringe 119a, and the plunger 119b have structures similar to those of the basal portion 101, the rear end portion 102, the branch tube 102b, the balloon 104, the front coil marker 105, the tubular passage 108, the syringe 109a, and the plunger 109b, respectively, of the balloon catheter 32.

The difference between the balloon catheter 32 and the balloon catheter 33 resides in the location of side openings 117 provided in the basal portion 111. Specifically, the basal portion 111 has a lumen 116 provided therein, through which the rear end of the branch tube 112b communicates with the side openings 117. The side openings 117 are provided in the basal portion 111 between the balloon 114 and the rear end of the basal portion 111. Therefore, even after the balloon 114 is inflated to plug the blood vessel, a drug solution can be fed through the lumen 116 to the region where the rear end of the basal portion exists and which is blocked by the balloon, or bodily fluid or other liquids can be removed from the region.

Different from typical balloon catheters used in the medical field, the balloon catheters 32 and 33 employed in the present invention have a basal portion containing both the tubular passage used for inflation of the balloon and the lumen used for administration of a drug solution. Use of either balloon catheter alone enables the operator to simultaneously inflate the balloon and administer a drug solution. Therefore, there is no need to insert two catheters into the body; i.e., a balloon catheter for blocking the blood vessel and a catheter for administration of a drug solution, thereby reducing the burden placed on the patient's body.

A balloon catheter 34 is used as a third balloon catheter in the embodiment according to the present invention. Different from the above-described balloon catheters 32 and 33 the balloon catheter 34 is a typical balloon catheter used in the field of the medicine. Specifically, the balloon catheter 34 has a basal portion, a rear end portion, and a balloon, but has no lumen for administration of a drug solution as provided in the balloon catheters 32 and 33.

The balloon catheters 32, 33, and 34 in the above-mentioned embodiment of the invention are constituted such that the balloon catheters themselves are inserted into the blood vessel without use of any guide wire. However, these balloon catheters may be of an overwire type, an RX type, or a monorail type, each of which uses a guide wire.

The introducers 30 and 31 and the balloon catheters 32, 33, and 34 are constituted as above. A balloon catheter is inserted into a blood vessel and indwelled at a predetermined position by use of an introducer; this step will next be described with reference to FIG. 1.

In this embodiment, a balloon catheter of the type such as the balloon catheter 32 shown in FIG. 7 is employed to block the celiac artery 12, the superior mesenteric artery 19, the inferior mesenteric artery 24, the right renal artery 20, and the left renal artery 21. Since the lumen 106 is provided inside the balloon catheter 32 so as to communicate with the tip side of the balloon 104, oxidized blood can be fed into the organ side through the lumen 106 after the balloon 104 is inflated.

In this embodiment, a balloon catheter of the type such as the balloon catheter 33 shown in FIG. 8 is employed to block the aorta 3. Since the lumen 116 is provided inside the balloon catheter 33 so as to communicate with the rear side of the balloon 114, a drug solution such as a contrast medium can be fed into the area where the vessels are blocked through the lumen 116 after the balloon 114 is inflated.

Further, in this embodiment, the balloon catheter 34 is employed to block the inferior vena cava 4. The balloon catheter 34 does not have a lumen and is used merely to block blood flow through inflation.

The balloon catheters 32, 33, and 34 are introduced into a living body through the non-branched sheath 40 or the three-way sheath 70. A method for percutaneously introducing the sheath used to insert the balloon catheter will next be described.

Firstly, a site of the skin above the target vessel for insertion is subjected to local anesthesia in preparation for introducing the non-branched sheath 40. After the effect of the anesthesia is confirmed, the anesthetized position is punctured by use of a known puncture needle or a similar needle. The guide wire 60 is then inserted into the vessel from the punctured portion and allowed to indwell at a predetermined position.

The rear end of the guide wire 60 is then inserted into the passage of the dilator 50, and the tip end of the non-branched introducer 30 is slowly inserted into the vessel percutaneously until the rear end of the guide wire 60 appears from the rear end of the dilator 50. The insertion of the non-branched introducer 30 is stopped when the tubular body 41 of the non-branched sheath 40 is located at a predetermined position in the vessel, and the non-branched sheath 40 is indwelled thereto.

Subsequently, the guide wire 60 is pulled out from the vessel to the outside of the body. The engagement between the thread 44a in the cap member 44 provided in the rear end portion 42 and the thread portion of the dilator 50 is released, and the dilator 50 is pulled out from the passage 45 of the non-branched sheath 40 to the outside of the body, to thereby indwell the tip end of the non-branched sheath 40 inside the body percutaneously. In this way, the tip end of the non-branched sheath 40 is inserted into the vessel.

In FIG. 1, the non-branched sheaths 40 are inserted into a living body. In this embodiment, the non-branched sheaths 40 are employed as sheaths to be inserted into the right axillary artery 9, the left axillary artery 11, the left femoral artery 6, and the right femoral vein 27. In FIG. 1, the non-branched sheaths 40 inserted into these vessels are represented by reference numerals 40-1, 40-2, 40-3, and 40-4.

The non-branched sheath 40-3 is placed inside the aorta 3 such that the tip end of the tubular body 41 is located around the lumbar arteries 25; an anticancer agent will be administered through the non-branched sheath 40-3.

The three-way sheath 70 is inserted into a vessel by means of the three-way introducer 31 through the same method employed for inserting the non-branched sheath 40 into a vessel. In this embodiment, the three-way sheath 70 is inserted through the right femoral artery 5.

The balloon catheters 32, 33, and 34 are introduced into a vessel through the non-branched sheath 40 or the three-way sheath 70 and indwelled at predetermined positions; this step will next be described.

In FIG. 1, reference numerals 32-1 and 32-2 denote the balloon catheters 32 introduced into a vessel through the non-branched sheaths 40-1 and 40-2, which are inserted into the right axillary artery 9 and the left axillary artery 11, respectively, and indwelled at the right renal artery 20 and left renal artery 21, respectively.

Reference numerals 32-3, 32-4, and 32-5 denote the balloon catheters 32 introduced into a vessel through the three-way sheath 70, which is inserted into the right femoral artery 5, and indwelled at the celiac artery 12, the superior mesenteric artery 19, and the inferior mesenteric artery 24, respectively.

The balloon catheter 33 is introduced into a vessel through the non-branched sheath 40-3, which is inserted into the left femoral artery 6, and indwelled in the aorta 3 at a predetermined position thereof. The balloon catheter 34 is introduced into a vessel through the non-branched sheath 40-4, which is inserted into the right femoral vein 27, and indwelled in the inferior vena cava 4 at a predetermined position thereof.

The balloon catheter 32-1 is introduced into a vessel through the non-branched sheath 40-1, which is inserted into the right axillary artery 9, and indwelled in the right renal artery 20 at a predetermined position thereof; the method for introducing and indwelling the catheter will next be described.

The balloon 104 of the balloon catheter 32-1 is initially in a deflated state. The tip end of the balloon catheter 32-1 is introduced into the passage 45 of the tubular body 41 through the small hole 43a provided in the hemostatic valve 43 of the non-branched sheath 40-1, and inserted into the right axillary artery 9.

The operator operates and guides the balloon catheter 32-1 while observing X-ray images obtained from fluororoentgenography such that the tip end is located at a predetermined position in the right renal artery 20, to thereby indwell the balloon catheter 32-1 therein.

The method described above may be employed to introduce the balloon catheter 32-2 into a vessel through the non-branched sheath 40-2, which is inserted into the left axillary artery 11, and to indwell the catheter in the left renal artery 21 at a predetermined position thereof. The method described above is also used to introduce the balloon catheters 32-3, 32-4, and 32-5 into vessels through the three-way sheath 70, which is inserted into the right femoral artery 5, and to indwell the catheters at predetermined positions in the celiac artery 12, the superior mesenteric artery 19, and the inferior mesenteric artery 24, respectively.

The method described above may also be employed to introduce the balloon catheter 33 into a vessel through the non-branched sheath 40-3, which is inserted into the left femoral artery 6, and to indwell the catheter in the aorta 3 at a predetermined position thereof. The method described above may also be employed to introduce the balloon catheter 34 into a vessel through the non-branched sheath 40-4, which is inserted into the right femoral vein 27, and to indwell the catheter in the inferior vena cava 4 at a predetermined position thereof.

No particular limitations are imposed on the insertion positions of catheters and balloon sheaths, but sheaths and balloon catheters are preferably inserted into different vessels such as the right axillary artery, the left axillary artery, the right femoral artery, the left femoral artery, and the right femoral vein as described above. This is because, when a plurality of sheaths or balloon catheters are introduced through the same vessel, the sheath or the catheter which has already been introduced may interfere with insertion of the next sheath or catheter. Also, passing of a plurality of sheaths or balloon catheters through a channel in a narrow blood vessel is likely to impose a burden on a patient and damage a sheath or catheter.

Preferably, the three-way sheath 70 is inserted through the femoral artery. The three-way sheath 70 has a large diameter, since three catheters are introduced therethrough. The femoral artery has a diameter large enough to pass the three-way sheath 70, and thus is preferable for introduction.

Preferably, the balloon catheter 33 is also inserted through the femoral artery. The balloon catheter 33 is used to block the aorta and has a larger diameter. The femoral artery has a diameter large enough to pass the balloon catheter 33 and thus is preferable for introduction.

A method for placing a tourniquet on the lower limb will next be described. In FIG. 1, reference numeral 35-1 denotes a tourniquet 35 applied to the right femoral region, and reference numeral 35-2 denotes a tourniquet 35 applied to the left femoral region.

A tourniquet comprises a flexible belt containing an inflatable tube, and pressurizing equipment connected to the tube as main components. In order to apply the tourniquet, the flexible tube is first rolled around the application area and firmly tied so as not to be loosened. The tourniquet 35-1 is applied to the right femoral region lower than the introduction positions of the three-way sheath 70 and the non-branched sheath 40-4. The tourniquet 35-2 is applied to the left femoral region lower than the introduction position of the non-branched sheath 40-3.

The tourniquet 35-1 inflates when air or liquid is fed into the tube contained therein from pressurizing equipment (not shown). Upon inflation, vessels in the mounted position of the tourniquet are stressed, to thereby block blood flow to/from arteries and veins in the right lower limb. This is also the case for the tourniquet 35-2, and blood flow to/from arteries and veins in the left lower limb is blocked when the tourniquet 35-2 is inflated.

The balloon 104 of the balloon catheter 32-1 is indwelled at a predetermined position in the right renal artery 20, and the plunger 109b of the syringe 109a is then pushed in, to thereby pass the liquid contained in the syringe 109a into the tubular passage 108 of the basal portion 101. The liquid in the tubular passage 108 is fed into the balloon 104 to inflate the balloon 104. The inflated balloon 104 tightly fits against the inner wall of the right renal artery 20 to form a blood flow blocked portion, whereby drug flow from the aorta 3 to the right kidney 22 is blocked.

The same method as above may be performed to block blood flow in the left renal artery 21 through inflation of the balloon 104 of the balloon catheter 32-2, whereby drug flow from the aorta 3 to the left kidney 23 is blocked.

The same method as above may be performed to block blood flow in the celiac artery 12 through inflation of the balloon 104 of the balloon catheter 32-3, whereby drug flow from the aorta 3 to the stomach 16, the liver 17, and the spleen 18 is blocked.

The same method as above may be performed to block blood flow in the superior mesenteric artery 19 through inflation of the balloon 104 of the balloon catheter 32-4, whereby drug flow from the aorta 3 to the small intestine is blocked.

The same method as above may be performed to block blood flow in the inferior mesenteric artery 24 through inflation of the balloon 104 of the balloon catheter 32-5. In this way, drug flow from the aorta 3 to the hindgut is blocked.

The same method as above may be performed to block blood flow at a predetermined position in the aorta 3 through inflation of the balloon 114 of the balloon catheter 33. In this way, backflow of a drug from downstream of the aorta 3 to the heart and the upper limb is prevented.

The same method as above may be performed to block blood flow at a predetermined position in the inferior vena cava 4 through inflation of the balloon of the balloon catheter 34. In this way, backflow of a drug from the aorta 3 to the inferior vena cava in the upper limb is prevented.

By means of the above steps, a blood-flow-blocked region can be provided in a region where blood flow is blocked by means of the tourniquets 35-1 and 35-2 and balloon inflation regions of the balloon catheters 32-1, 32-2, 32-3, 32-4, 32-5, 33, and 34.

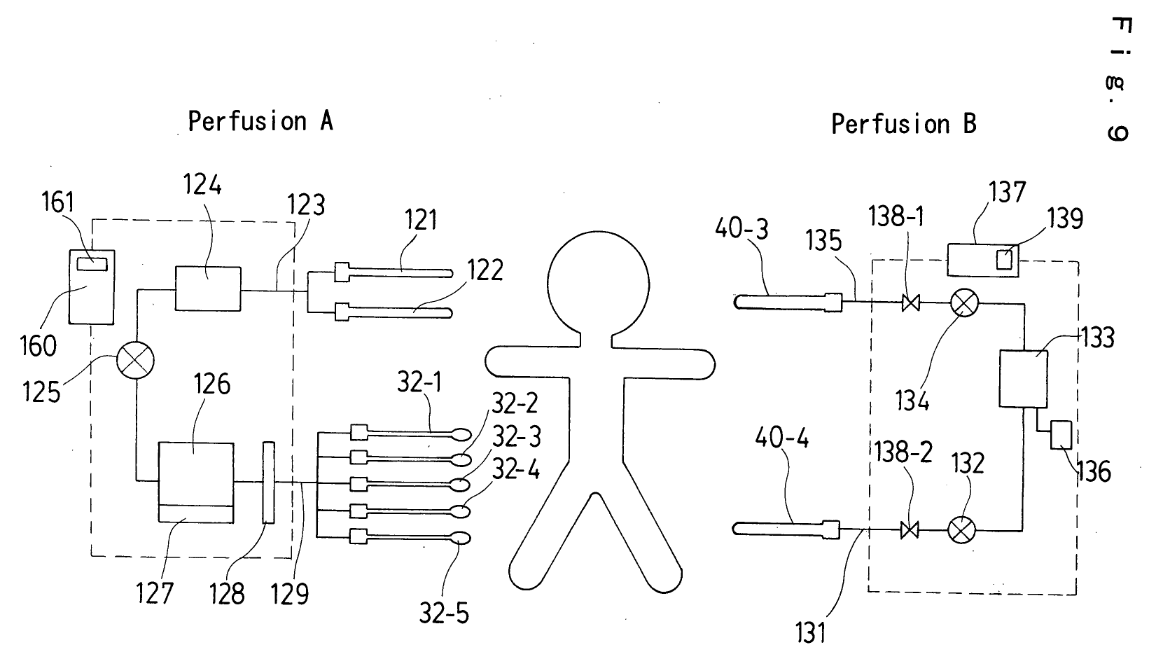

FIG. 9 is an illustration showing the configuration of a perfusion circuit according to an embodiment of the present invention. A method for administering a drug in the aorta blocked through the above technique will next be described. In this embodiment, two types of perfusion are performed. One is performed to protect organs (organ-protecting perfusion A), and the other is performed to administer a drug such as an anticancer agent (drug-administering perfusion B).

(Organ-Protecting Perfusion A)

In this type of perfusion, venous blood drained from the superior and inferior vena cavas is oxygenated by means of an artificial lung, and the oxygenated blood is fed into the right renal artery 20, the left renal artery 21, the celiac artery 12, the superior mesenteric artery 19, and the inferior mesenteric artery 24. This type of perfusion is performed in order to protect organs, in which blood supply is blocked by inflation of balloons, through feeding of oxygenated blood.

The configuration of equipment for performing the organ-protecting perfusion comprises venous cannulas 121 and 122 for removal of blood, a drainage tube 123, a reservoir 124, a pump 125, an artificial lung 126, a heat exchanger 127, a filter 128, a feeding tube 129, and balloon catheters 32-1, 32-2, 32-3, 32-4, 32-5.

The venous cannulas 121 and 122 are inserted through the jugular by means of a known method and indwelled in the superior vena cava and the inferior vena cava, respectively. Venous blood is drained to the outside of the body by means of the two cannulas and, via the drainage tube 123, stored in the reservoir 124. A flow sensor or a similar sensor may be provided between the cannulas 121, 122 and the reservoir 124. The venous blood stored in the reservoir 124 is fed into the artificial lung 126 by means of the pump 125. A known pump such as a roller pump or a centrifugal pump may be employed as the pump 125.

The venous blood fed into the artificial lung 126 undergoes gas exchange of carbon dioxide and oxygen, to thereby produce oxygenated blood containing a large amount of oxygen. In the artificial lung 126, gases are exchanged by use of a membrane such as a hollow fiber membrane or a film membrane. The artificial lung 126 is equipped with the heat exchanger 127. The heat exchanger 127 provides temperature control of blood. In the heat exchanger 127, blood temperature is controlled by warm or cold water through a medium having a high heat conductivity such as a metal plate.

The oxygenated blood gas-exchanged in the artificial lung 126 is filtrated by means of the filter 128 and then returned to arteries. In the filter 128, foreign substances such as blood clots and air bubbles are removed. The oxygenated blood passing through the filter 128 is fed into arteries through the feeding tube 129 and then the balloon catheters 32-1, 32-2, 32-3, 32-4, and 32-5.

The oxygenated blood passing through the balloon catheter 32-1 is fed into the right renal artery 20 through the lumen 106 and supplied to the right kidney 22. The oxygenated blood passing through the balloon catheter 32-2 is fed into the left renal artery 21 through the lumen 106 and supplied to the left kidney 23.

The oxygenated blood passing through the balloon catheter 32-3 is fed into the celiac artery 12 through the lumen 106 and supplied to the stomach 16, the liver 17, and the spleen 18. The oxygenated blood passing through the balloon catheter 32-4 is fed into the superior mesenteric artery 19 through the lumen 106 and supplied to the small intestine. The oxygenated blood passing through the balloon catheter 32-5 is fed into the inferior mesenteric artery 24 through the lumen 106 and supplied to the hindgut.

In another conceivable embodiment, oxygenated blood is fed into any one or two catheters selected from the balloon catheters 32-3, 32-4, and 32-5 and is not fed into the other catheters.

As shown in FIG. 2, the common hepatic artery 14 and the superior mesenteric artery 19 communicate through the first collateral pathway 29a extending therebetween, and the superior mesenteric artery 19 and the inferior mesenteric artery 24 communicate through the second collateral pathway 29b extending therebetween. Therefore, oxygenated blood is not necessarily fed into all of the three arteries; i.e., the common hepatic artery 14, the superior mesenteric artery 19, the inferior mesenteric artery 24, and oxygenated blood can be supplied to all of the above three arteries by feeding only one or two arteries. In this embodiment, a complicated operation that oxygenated blood is fed into the all above three arteries is unnecessary.

In particular, oxygenated blood is preferably fed only into the balloon catheter 32-4 inserted into the superior mesenteric artery 19. Since the small intestine may be greatly damaged when an insufficient amount of oxygen is supplied, oxygenated blood must be preferentially fed into the superior mesenteric artery 19, which communicates with the small intestine. Meanwhile, the other organs may not be greatly damaged even when blood supply is interrupted for a short period of time, and oxygenated blood supplied through the collateral pathways 29a and 29b may be sufficient to protect the organs.

In this case, a balloon catheter into which oxygenated blood is not fed is not necessarily a balloon catheter having a lumen such as the balloon catheter 32, and may be a conventional balloon catheter without a lumen such as the balloon catheter 34.

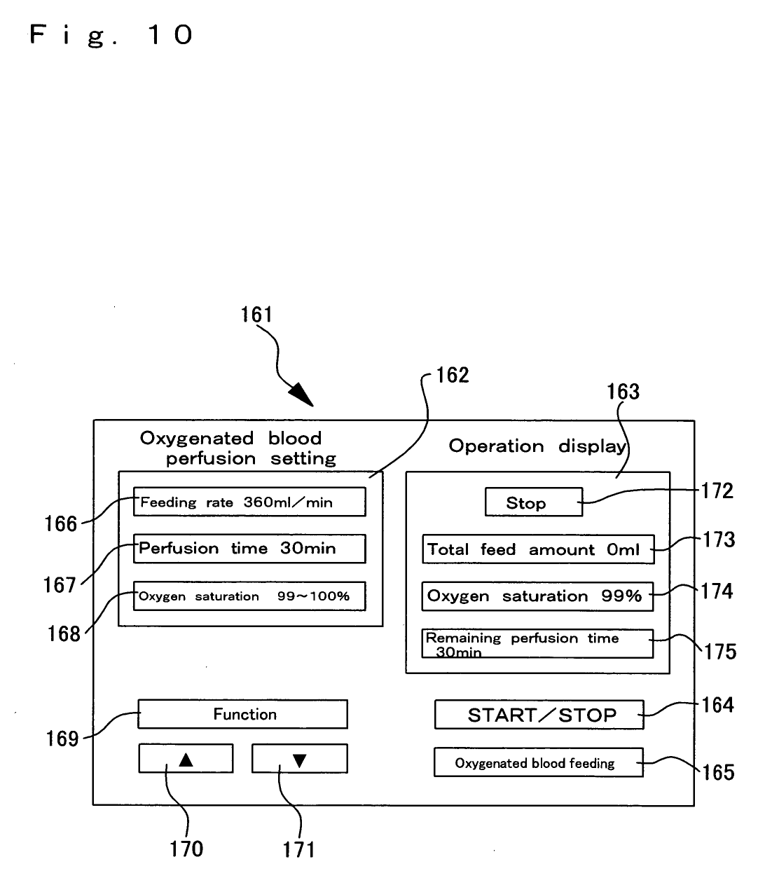

FIG. 10 is an illustration showing a settings display panel 161 of a controller 160 of the embodiment of the present invention employed in perfusion A. The settings display panel 161 is a screen for presetting various values of a perfusion apparatus used in perfusion A and for confirming the operating conditions, and comprises an oxygenated blood perfusion settings display 162, an operation display 163, a running/stopped display 164, and an oxygenated blood feeding key 165.

The oxygenated blood perfusion settings display 162 comprises a feed rate settings display 166 for displaying the “feed rate set value” of oxygenated blood, a perfusion time settings display 167 for displaying the “perfusion time set value,” and an oxygen saturation settings display 168 for displaying the “oxygen saturation set value” (i.e., the oxygen saturation (SO₂) in blood).

A function key 169 is used to sequentially switch, by pushing the key, a display to which set value can be input, between the feed rate settings display 166, the perfusion time settings display 167, and the oxygen saturation settings display 168. Set values can be continuously changed by means of an up key 170 and a down key 171.

Signals corresponding to the preset values of the feed rate settings display 166, the perfusion time settings display 167, and the oxygen saturation settings display 168 are transmitted to the controller 160, and the controller 160 can start and control the pump 125 to achieve predetermined operations such that a predetermined amount of blood or other fluid is perfused within a predetermined period of time.

In response to operation of the oxygenated blood feeding key 165 at any time during operation, a signal corresponding to the “feed rate set value” displayed on the feed rate settings display 166 is transmitted to the controller 160, and the controller 160 can start and control the pump 125 to achieve predetermined operations.

When the running/stopped display 164 is pressed at startup, a signal corresponding to the “feed rate set value” displayed on the feed rate settings display 166 is transmitted to the controller 160, and the controller 160 starts and controls the pump 125 to achieve predetermined operations.

The operation display 163 displays the states of the respective operations during running and includes a running/stopped display 172 for displaying a running or stopped state, a total feed amount display 173 for displaying the total amount of oxygenated blood fed, an oxygen saturation display 174 for displaying oxygen saturation, and a remaining perfusion time display 175 for displaying the remaining time of perfusion. The value displayed on the total feed amount display 173 is calculated from a value measured by means of a flow sensor or similar sensor.

Operation of the controller 160 will next be described. An operator sets the “feed rate set value,” the “perfusion time set value,” and the “oxygen saturation set value” by use of the settings display panel 161. Thereafter, the anticancer agent perfusion apparatus dotted a line in FIG. 9 is activated by pressing of the running/stopped display 164. Specifically, the controller 160 starts and controls the pump 125 and the artificial lung 126 on the basis of the set values displayed on the oxygenated blood perfusion settings display 162. At this point in time, the running/stopped display 164 is changed from “stop” to “start.”

As shown in FIG. 9, venous blood is first stored in the reservoir 124 via the cannulas 121 and 122 and then the drainage tube 123 through the suction and discharge operations of the pump 125.

The venous blood stored in the reservoir 124 is fed into the artificial lung 126 through operation of the pump 125. In the artificial lung 126, the venous blood is subjected to oxygenation under the control by the controller 160. An dissolved oxygen sensor is provided in the artificial lung 126, and the controller 160 controls the operation of the artificial lung 126 in a feedback manner according to the measurements of the dissolved oxygen sensor such that the oxygen saturation in the oxygen saturation settings display 168 is maintained.

The oxygenated venous blood is fed into the feeding tube 129 through the suction and discharge operations of the pump 125. The oxygenated blood passes through the filter 128 and is fed inside the body through the balloon catheters 32-1, 32-2, 32-3, 32-4, and 32-5 at a predetermined flow rate adjusted by means of a feeding pump.

When the oxygenated blood perfusion is applied for a predetermined period of time, a counter (not shown) provided in the controller 160 is activated, and the controller 160 stops the pump 125 and the artificial lung 126. At this point in time, the running/stopped display 172 is changed from “start” to “stop.”

(Perfusion B for Administering A Drug)

In the perfusion B, a drug solution containing an anticancer agent is administered through the non-branched sheath 40-3, and a drug-containing bodily fluid is collected through the non-branched sheath 40-4.

In the perfusion B, circulation is performed through a circuit containing the non-branched sheath 40-3, the non-branched sheath 40-4, a discharge tube 131, a discharge pump 132, a reservoir 133, a feeding pump 134, a feeding tube 135, a drug feeder 136, a controller 137, flow rate sensors 138-1 and 138-2, and a settings display panel 139.

The three-way cock 48 of the non-branched sheath 40-4 is connected to the discharge tube 131. The discharge tube 131 is connected to the reservoir 133. The discharge tube 131 is provided with the flow rate sensor 138-2 and the discharge pump 132.

Blood is drawn from the inferior vena cava under suction generated by the discharge pump 132, and then flows sequentially through the passage 45, the side port 46, the flexible tube 47, and the three-way cock 48 of the non-branched sheath 40-4 into the discharge tube 131. Subsequently, the blood is delivered to the reservoir 133. The discharge pump 132 employed in the present invention may be a known pump such as a roller pump or a centrifugal pump.

The discharge tube 131 is connected with the reservoir 133, which is connected to the drug feeder 136. The drug feeder 136, which is an apparatus for feeding an anticancer agent into the discharge tube 131, contains a syringe pump and is controlled by the controller 137.

The syringe pump has a worm gear structure or a ball screw structure, and is able to feed a predetermined amount of a drug contained in the syringe by maintaining the syringe body in a stationary state and moving a piston by a predetermined distance through use of a slider. The anticancer agent stored in the reservoir 133 is drawn under suction made by the feeding pump 134 and delivered into the body by means of the pump 134.

One or more additional feeders containing a necessary agent such as infusion liquid, saline, or an anticoagulant may be provided in the blood circuit in a manner similar to that of the drug feeder 136, for adding the necessary agent to the circuit of the perfusion B in a predetermined amount. Thus, proportions of necessary agent, anticancer agent, and blood can be set or controlled arbitrarily.

The flow rate sensor 138 may include a Doppler ultrasound flow meter, which enables measurement of volumes of blood collected from or delivered into the body without coming into direct contact with the liquid to be measured containing an anticancer agent, blood, or others. Other ultrasound-based flow rate measuring methods include, in addition to the Doppler method, the time difference method and sing-around method. Any of these methods may be employed to construct a sensor.

The flow rate sensor 138 has a transmitting piezoelectric element (not shown) which generates ultrasonic waves into the flow, and a receiving piezoelectric element (not shown) which receives a Doppler signal. The transmitting piezoelectric element transmits ultrasonic waves to the flow containing blood flowing through the feeding tube 135 or the discharge tube 131. The receiving piezoelectric element receives a Doppler signal corresponding to the flow rate of the blood-containing flow.

The received signal is amplified in an amplifier (not shown). High-frequency components of the resultant signal are removed by a low-pass filter, and then transmitted to the controller 137 as a measurement value corresponding to the flow rate.

In the embodiment shown, the flow rate sensors 138-1 and 138-2 are disposed around the feeding tube 135 and the discharge tube 131, respectively. Alternatively, the flow rate sensor 138-1 may be disposed in a flow channel (not shown) within the feeding pump 134 in place of the surroundings of the feeding tube 135. Similarly, the flow rate sensor 138-2 may be disposed in a flow channel (not shown) within the discharge pump 132 in place of the surroundings of the discharging tube 131.

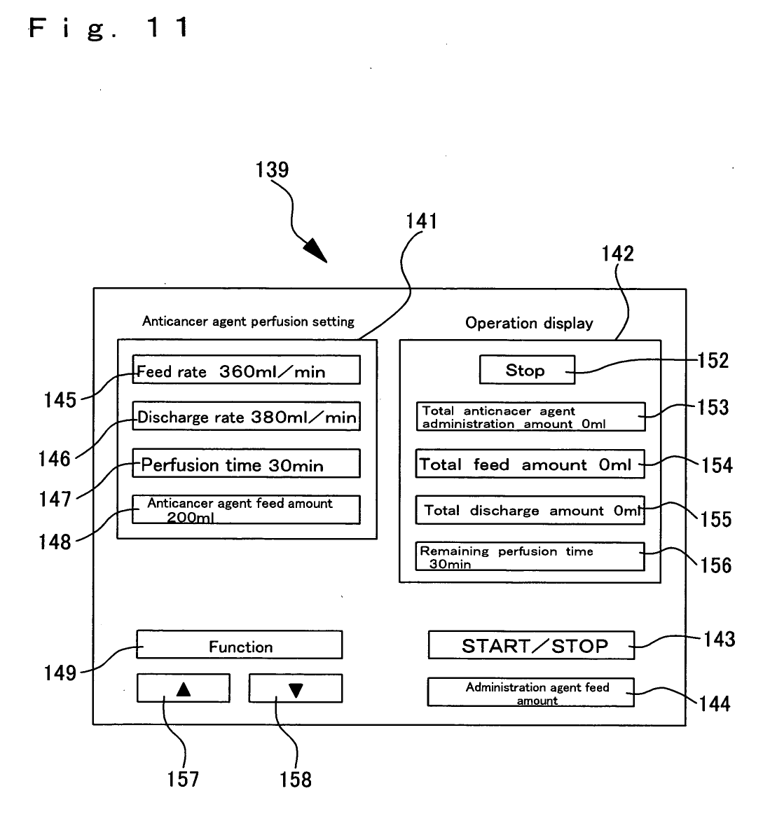

FIG. 11 shows the settings display panel 139 of the controller 137 employed in the perfusion B. The settings display panel 139 is a screen for presetting various values for performing the anticancer agent perfusion therapy and for confirming the operating conditions. The settings display panel 139 comprises an anticancer agent perfusion settings display 141, an operation display 142, an start/stop switch 143, and an anticancer agent administration key 144.

The anticancer agent perfusion settings display 141 comprises a feed rate settings display 145 for displaying the “feeding rate set value” of the anticancer agent-containing blood or the like, a discharge rate settings display 146 for displaying the “discharge rate set value” of the blood or the like, a perfusion time settings display 147 for displaying the “perfusion time set value,” and a anticancer agent feeding amount settings display 148 for displaying the “the anticancer agent amount set value” representing the amount of the agent to be fed into the body within a time period of an operation.

A function key 149 is used to sequentially switch, by pushing the key, a display to which set value can be input, between the feed rate settings display 145, the discharge rate settings display 146, the perfusion time settings display 147, and the anticancer agent feed amount settings display 148. Set values can be continuously changed by means of an up key 157 and a down key 158.

Signals corresponding to the set values of the feed rate settings display 145, the discharge rate settings display 146, and the perfusion time settings display 147 are transmitted to the controller 137, and the controller 137 can start and control the feeding pump 134 and the discharge pump 132 to achieve predetermined operations such that a predetermined amount of blood or other fluid is perfused within a predetermined period of time.

By operating the anticancer agent administration key 144 at any time during operation, a signal corresponding to the “the anticancer agent feed amount set value” displayed on the anticancer agent feed amount settings display 148 is transmitted to the controller 137, and the controller 137 can start and control the drug feeder 136 to achieve predetermined operations.

By pressing the start/stop switch 143 at startup, a signal corresponding to the “anticancer agent perfusion time set value” displayed on the anticancer agent perfusion time settings display 148 is transmitted to the controller 137, and the controller 137 starts and controls the drug feeder 136 to achieve predetermined operations.

The operation display 142 displays the states of the respective operations during running and includes a running/stopped display 152 for displaying a state of running or being stopped, a total anticancer agent feed amount display 153 for displaying the total amount of anticancer agent fed, a total feed amount display 154 for displaying the total amount of blood or other fluid fed, a total discharge amount display 155 for displaying the total amount of blood or other fluid discharged, and a remaining perfusion time display 156 for displaying the remaining time of perfusion.

The displayed value on the total anticancer agent feed amount display 153, indicating the total amount of the anticancer agent fed, is calculated by use of the “feed amount set value” displayed on the anticancer agent feed amount settings display 148 together with the number of times the anticancer agent administration key 144 is pressed. The displayed value on the total feed amount display 154 is calculated from the value measured by means of the flow rate sensor 138-1. The displayed value on the total discharge amount display 155 is calculated from the value measured by means of the flow rate sensor 138-2.

The operation of the anticancer agent perfusion apparatus will next be described. An operator sets the “feed rate set value,” the “discharge rate set value,” the “perfusion time set value,” and the “anticancer agent feed amount set value” by use of the settings display panel 139. Thereafter, the anticancer agent perfusion apparatus dotted a line in FIG. 9 is activated by switch-on of the start/stop switch 143. Specifically, the controller 137 starts and controls the feeding pump 134, the discharge pump 132, and the drug feeder 136 in accordance with the set values displayed on the anticancer agent perfusion settings display 141. At this point in time, the running/stopped display 152 is changed from “stop” to “running.”

As shown in FIG. 9, firstly, the anticancer agent contained in the drug feeder 136 is introduced into the blood circuit. The anticancer agent is delivered to the feeding tube 135 by means of the pump 134.

The anticancer agent has been stored in advance in the reservoir 133 in the form of a mixture with saline, the mixture having an appropriate anticancer agent concentration. The anticancer agent contained in the reservoir 133 is delivered into the feeding tube 135 by means of the feeding pump 134. Subsequently, the anticancer agent is delivered through the non-branched sheath 40-3 into the body at a predetermined flow rate adjusted by means of the feeding pump 134.

Specifically, the flow rate sensor 138-1 measures the flow rate of the anticancer agent delivered through the feeding tube 135 and transmits a signal corresponding to the flow rate to the controller 137. If the deviation of the flow rate value reflected by the signal from the “feeding rate set value” is greater than a predetermined threshold, the controller 137 controls the feeding pump 134 in a feedback manner, whereby the flow rate is recovered and maintained to the “feeding rate set value.”

When the feeding pump 134 is activated, the anticancer agent fed through the feeding tube 135 is delivered intracorporeally by way of perfusion. Bodily fluid containing the anticancer agent is delivered through the non-branched sheath 40-4 into the discharging tube 131. The bodily fluid is delivered by means of the discharge pump 132 into the reservoir 133.

To the bodily fluid contained in the reservoir 133, the anticancer agent is added by means of the drug feeder 136. A sensor (not shown) is provided in the reservoir 133 for measuring the concentration of the anticancer agent contained therein, and the anticancer agent is added to the reservoir 133 until the resultant mixture has an appropriate anticancer agent concentration.

The bodily fluid such as blood (hereinafter may be referred to simply as “blood”) is delivered to the feeding tube 135 by means of the feeding pump 134, and the fluid is fed into the body through the non-branched sheath 40-3 at a flow rare of 200 mL/min adjusted by means of the feeding pump 134.

The flow rate sensor 138-2 measures the flow rate of the blood delivered into the discharge tube 131 and transmits a signal corresponding to the flow rate to the controller 137. The controller 137 controls the discharge pump 132 in a feedback manner in accordance with the signal reflecting the flow rate so that the flow rate is maintained to the “discharge rate set value.”

By activating the anticancer agent administration key 144 at an arbitrary but appropriate time during the operation, the anticancer agent is introduced into the blood circuit by means of the drug feeder 136 in an amount (200 mL) displayed on the anticancer agent feed amount settings display 148.

The anticancer agent introduced to the blood circuit is mixed with the blood contained in the reservoir 133 by means of the feeding pump 134. Thus, the blood containing the anticancer agent is again fed into the body through the non-branched sheath 40-3 at a predetermined flow rate adjusted by means of the feeding pump 134.

The operator may change, during operation, the amount of the anticancer agent fed into the body or may determine the number of times the anticancer agent should be fed, based on the anticancer agent content of the blood determined through a known technique such as centrifugation of the blood received in the reservoir 133 or urinalysis.

When the anticancer agent perfusion is applied for a predetermined period of time, a counter (not shown) provided in the controller 137 is activated, and the controller 137 stops the feeding pump 134, the discharge pump 132, and the drug feeder 136. At this point in time, the running/stopped display 152 is changed from “running” to “stopped.”

A drug employed in the present invention includes not only drugs used for therapy of cancers in chemotherapy, immunotherapy, a therapy using a radioactive isotope, gene therapy, or a similar therapy, but also agents, such as antihemolytic agents, used in preliminary treatments for cancer therapy.

Chemotherapy is a method for treating cancer through destruction of tumor cells by use of drugs such as anticancer agents. Anticancer agents are typically classified into antimetabolites, alkylating agents, anticancer antibiotics, and vegetable alkaloids. Antimetabolites inhibit cell division of rapidly growing cancer cells through use of enzymes contained in a large amount in the cancer cells. Alkylating agents, which have been developed in the form of toxic gas, react with and destroy DNA—which plays an important role in essential life activities such as transfer of genetic information—by binding thereto, to thereby kill cancer cells.

Similar to typical antibiotics, anticancer antibiotics are generally produced from microorganisms contained in the soil. Vegetable alkaloids inhibit the function of microtubules, which are important to cell division, to thereby kill cancer cells. Examples of such an anticancer agent which acts on the microtubules include paclitaxel.

Immunotherapy is another method for treating cancer in which tumor tissues are killed through enhancement of immunofunction of a patient. Examples of drugs employed for enhancing immunofunction include cytokines, vaccines, and immunocytes of the patient himself/herself. A cytokine, a substance produced by immunocompetent cells, activates or proliferates immunocompetent cells. The activated immunocytes directly or indirectly kill tumor cells.

A vaccine is a substance produced through detoxication (e.g., destruction) of tumor cells of a patient, and the thus-treated cells are inoculated to the patient. Through the vaccine inoculation, immunoreaction against a cancer-specific antigen is induced in the patient's body, to thereby kill tumor cells. Examples of the immunocytes of patient include lymphocyte and dendritic cells. These cells are collected from the patient, cultured ex vivo, and returned to the patient's body. Such immunotherapy is generally said to cause less intense side effect than does chemotherapy.

In the present invention, as a radioactive isotope, there may be employed an element such as ¹²³I, ¹²⁵I, ¹³¹I, ³²P, ⁶⁴Cu, ²¹¹At, ¹⁷⁷Lu, ⁹⁰Y, ¹⁸⁶Re, ²¹²Pb, or ²¹²Bi. Alternatively, a known tumor-specific antigen labeled with any of the radioactive isotopes may be employed. Such a radioactive isotope directly acts on DNA of tumor cells to render the cells no longer able to divide and to induce apoptosis, to thereby kill the tumor cells. The radioactive isotope is mixed with, for example, saline before use.

As a gene, DNA molecule, RNA molecule, or cell for gene therapy, a substance generally employed in gene therapy of cancer may be employed. The gene, DNA molecule, RNA molecule, or cell may be employed singly or in combination with one or more other drugs, to inhibit functions of a cancer gene, recover functions of an inactivated cancer suppressor gene, or enhance the immunological competence against tumors. Such a gene, DNA molecule, RNA molecule, or cell is used in the form of a mixture with, for example, saline.

As a gene for gene therapy, a cancer suppressor gene, a drug metabolizing enzyme gene, or a DNA vaccine may be employed. The cancer suppressor gene employed in the present invention is exemplified by a p53 gene. When the p53 gene is introduced into mutated cancer cells in which cancer suppressor genes have been inactivated, the p53 gene lowers oncogenicity of the cells.

A drug metabolizing enzyme gene is a gene employed in suicide gene therapy and is derived from a microorganism which, in nature, is not present in cells of mammals. The metabolizing enzyme gene selectively kills only cells to which the metabolizing enzyme gene is introduced, by introducing into cells the metabolizing enzyme gene and administering a prodrug which is transformed into an active (toxic) product by an enzyme encoded by the gene. The prodrug is typically an antimicrobial agent such as an antivirus or an antifungal agent.

As a combination of the drug metabolizing enzyme gene and the prodrug, there may be employed a combination of herpes simplex virus thymidine kinase (HSV-TK), and ganciclovir (GCV) or aciclovir (ACV); a combination of cytosine deaminase and 5-fluorocytosine (5-FC); a combination of varicella zoster virus thymidine kinase and 6-methoxypurine arabinoside (ara-M); a combination of E. coli gpt and 6-thioxanethine (6-TX); and a combination of E. coli deoD and 6-methylpurine-2′-deoxyriboside (Mep-dR).

As a DNA molecule for gene therapy, an antisense oligodeoxynucleotides (antisense ODN) may be employed in the present invention. An antisense oligodeoxynucleotide is a small DNA molecule which has a base sequence complementary to mRNA of the target cancer, and inhibits expression of the target cancer gene by hybridizing the target mRNA in cells and inhibiting translation to proteins.

As a RNA molecule for gene therapy, an antisense RNA or a ribozyme may be employed in the present invention. The antisense RNA is expressed by an antisense gene (cDNA) and inhibits expression of the target cancer gene. The ribozyme is an RNA molecule that has the ability to cleave the complementary mRNA strands, and is formed from an antisense portion against the target mRNA and an active portion exhibiting the cleavage activity. The active portion cleaves mRNA of the target cancer gene and inhibits translation to proteins, to thereby suppress expression of the target gene. In embodiments of the present invention, any of hammerhead type, hairpin type, and hepatitis D type ribozymes may be employed.

As a cell for gene therapy, an effector cell into which a cytokine gene is introduced may be employed in the present invention. The cytokine gene may be tumor necrosis factor (TNF), interferon-γ (INF-γ), FCR, TCR, IL-2R, IL-2, IL-7, or IL-12. The effector cell may be tumor infiltrating lymphocyte (TIL), LAK cell, or cytotoxic T cell (CTC).

The gene, DNA molecule, RNA molecule, and cell for gene therapy are not limited to those described above, and other genes, DNA molecules, RNA molecules, and cells may be employed.

The term “agents used in preliminary treatments for cancer therapy” refers to substances which are used in operation, treatment, or diagnosis of cancer but which themselves have no effect of directly or indirectly killing tumors. Examples of the agents used in preliminary treatments for cancer therapy include antihemolytic agents such as haptoglobulin, which is used to prevent hemolysis of the blood vessels during insertion of catheter. Examples of the agents used in diagnosis of cancer include iodine contrast agents employed in angiography.

The bodily fluid collected from the body may be any bodily fluid contained in the body, including blood and lymph.

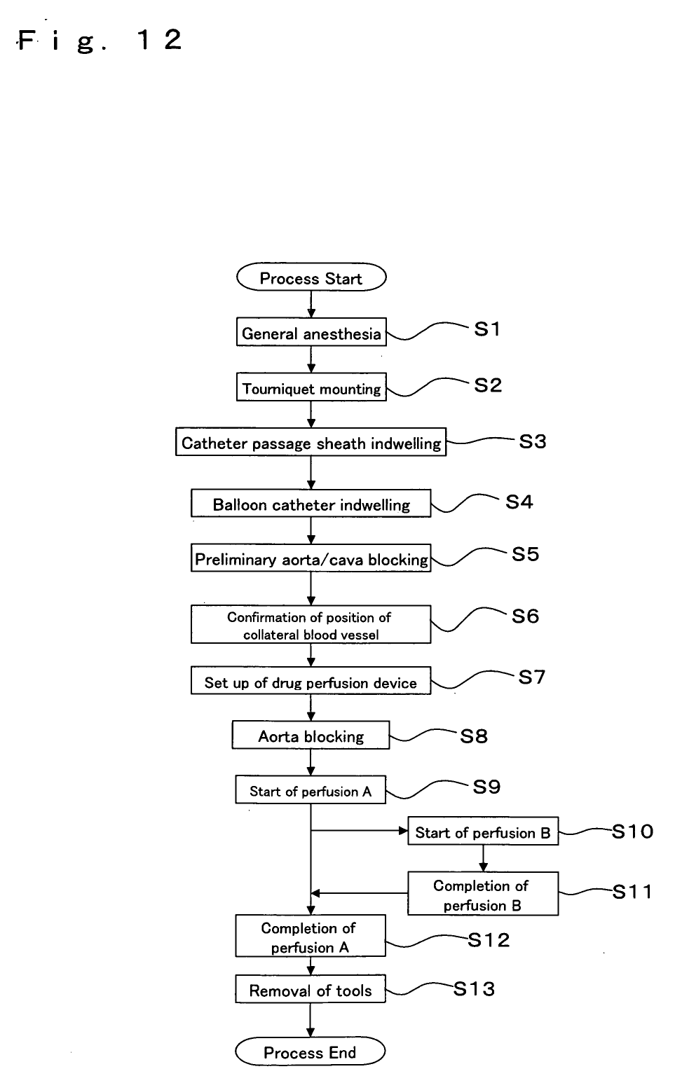

Now, with reference to the flow chart shown in FIG. 12, the technique and procedure of the drug perfusion therapy according to the invention will be described. The procedure described below was decided to be employed, in view that the occurrence of advanced cancer in a site within the paraaortic lymph nodes 26 was clear from laboratory tests and that the test results did not support surgical operation, but intervention by way of perfusion. The mode of this embodiment is shown in FIG. 1.

In Step S1, a patient is generally anesthetized in a routine manner.

In Step S2, tourniquets 35 are placed. In this step, the tourniquets 35 are kept in a loosened state so that the blood flow in the lower limbs is not blocked.

In Step S3, sheaths are indwelled at predetermined locations in the blood vessels. Specifically, tips of non-branched sheaths 40-1, 40-2, 40-3, 40-4, and three-way sheath 70 are percutaneously inserted and indwelled in the right axillary artery 9, the left axillary artery 11, the left femoral artery 6, the right femoral vein 27, and the right femoral artery 5, respectively.

In Step S3, blood-removing cannulas 121 and 122 employed in the process of perfusion A are also percutaneously inserted through the jugular veins and indwelled at the superior vena cava and the inferior vena cava, respectively, through a known method.

In Step S4, balloon catheters are indwelled at predetermined locations in the blood vessels. Specifically, a balloon catheter 32-1 is inserted through the non-branched sheath 40-1 into the right axillary artery 9, and the balloon-inflating portion thereof is indwelled at a predetermined location in the right renal artery 20. A balloon catheter 32-2 is inserted through the non-branched sheath 40-2 into the left axillary artery 11, and the balloon-inflating portion thereof is indwelled at a predetermined location in the left renal artery 21. Balloon catheters 32-3, 32-4, and 32-5 are inserted through the three-way sheath 70 into the right femoral artery 5, and the balloon-inflating portions thereof are indwelled at predetermined locations in the celiac artery 12, the superior mesenteric artery 19, and the inferior mesenteric artery 24.

A balloon catheter 33 is inserted through the non-branched sheath 40-3 into the left femoral artery 6, and the balloon-inflating portion thereof is indwelled at a predetermined location in the aorta 3 between the celiac artery 12 and the heart. A balloon catheter 34 is inserted through the non-branched sheath 40-4 into the right femoral vein 27, and the balloon-inflating portion thereof is indwelled at a predetermined place in the inferior vena cava 4.

Prior to controlling the balloon catheters 32, 33, and 34 under X-ray fluoroscopy, syringes containing a contrast medium have been connected to three-way cocks of the non-branched sheath 40 and the three-way sheath 70. If a branched portion is present in the blood vessel, the radiopaque contrast medium is injected into the blood vessel by means of pressurizing the syringe, and the balloon-inflating portion is selectively advanced into a suitable one of the branches while performing angiography through an appropriate level of X-ray irradiation.

In Step S5, preliminary cava blocking is carried out. Specifically, the tourniquets 35 placed in Step S2 are inflated by applying an appropriate pressure to thereby block the blood flow in the lower limbs. In addition, preliminary aorta/cava blocking is performed in the aorta and the cava by applying an appropriate pressure to the balloon catheters 32-1, 32-2, 32-3, 32-4, 32-5, 33, and 34 indwelled in Step S4, thereby inflating the balloon-inflating portions thereof.

In Step S6, positions of collateral blood vessels are confirmed. Specifically, a radiopaque contrast medium is introduced to the blood vessel, and angiography is performed through an appropriate level of X-ray irradiation, to thereby determine whether collateral blood vessels are present or absent in the regions of the above-mentioned preliminary aorta/cava blocking and to specify the sites. The radiopaque contrast medium is fed through the lumen 116 of the balloon catheter 33. Specifically, a syringe containing the contrast medium is attached to the branch tube 112b of the balloon catheter 33, and the plunger of the syringe is pushed, to thereby feed the contrast medium. The contrast medium is fed to the blood vessel via the side openings 117.

After confirmation that collateral blood vessels to and from tumor tissues have been secured, and that therefore drugs can reach the tumor tissues, the tourniquets 35 and balloons are immediately deflated to thereby restore the blood flow.

In Step S7, a drug perfusion apparatus is set. The perfusion method of the present invention employs two types of perfusion; i.e., perfusion A and perfusion B, as shown in FIG. 9. A liquid-feeding tube 135 is connected to one of the ports provided in the three-way cock 48 of the non-branched sheath 40-3. In addition, a syringe (not shown) containing necessary agents, including an anticoagulant (such as heparin) and a contrast medium, is connected to the branch tube 112b of the balloon catheter 33.

The following parameters are set for perfusion A by means of the settings display panel 161 shown in FIG. 10: the “liquid feed rate setting” displayed on the liquid feed rate settings display 166 is set at 360 mL/min; the “perfusion time setting” displayed on the perfusion time settings display 167 is set at 30 min; and the “oxygen saturation setting” displayed on the oxygen saturation settings display 168 is set at 99 to 100%.

The following parameters are set for perfusion B by means of the settings display panel 139 shown in FIG. 11: the “liquid feed rate setting” displayed on the liquid feed rate settings display 145 is set at 200 mL/min; the “liquid discharge rate setting” displayed on the liquid discharge rate settings display 146 is set at 180 to 200 mL/min; and the “perfusion time setting” displayed on the perfusion time settings display 147 is set at 30 min.

In Step S8, aorta/cava blocking is performed. Specifically, while appropriately feeding an anticoagulant such as heparin, the tourniquets 35 are inflated by pressurizing at an appropriate pressure, to thereby block the blood flow in the lower limbs. The balloons of the balloon catheters 32-1, 32-2, 32-3, 32-4, 32-5, 33, and 34 are also inflated by pressurizing at an appropriate pressure the syringes mounted at the rear end of the catheters, to thereby form a closed region in the aorta/cava.

In Step S9, the perfusion A is carried out by actuating the running/stopped display 164 provided on the settings display panel 161. Specifically, oxygenated blood is fed to the right kidney, the left kidney, the stomach 16, the liver 17, the spleen 18, the small intestine, and the hindgut through the lumen 116 of the balloon catheters 32-1, 32-2, 32-3, 32-4 and 32-5.

In Step S10, the perfusion B is started by activating the start/stop switch 143 provided on the settings display panel 139 while operation of the perfusion A is maintained. Specifically, a drug solution containing an anticancer agent is administered to the aorta/cava closed region through the passage 45 of the non-branched sheath 40-3. In addition, bodily fluid containing the anticancer agent is removed from the aorta/cava closed region through the passage 45 of the non-branched sheath 40-4.

During the above anticancer agent perfusion procedure, the anticancer agent is administered to the cancer tissues by actuating the anticancer agent administration key 144 at 10-minute intervals. In the present embodiment, the dose of the anticancer agent, the times of anticancer agent administration, and the frequency of anticancer agent administration can be modified depending on conditions of the patient or based on the anticancer agent content of the blood as determined by, for example, centrifugation of the blood stored in the reservoir 133 or urinalysis.

At an arbitrary, appropriate time, a haptoglobulin-containing antihemolytic agent may desirably be administered intravenously. In addition, blood circulating conditions are preferably maintained, by providing blood transfusion or fluid transfusion during operation.

After an appropriate amount of an anticancer agent has been administered predetermined times, the running/stopped display 152 automatically changes to thereby complete the process of perfusion B (Step S1).

Thereafter, the process of perfusion A is completed by operating the running/stopped display (Step S12). Subsequently, the tourniquets 35 and the balloon-inflating portions of the balloon catheters 32, 33, and 34 are deflated, to thereby release the aorta/cava blocking. An appropriate amount of a heparin antagonist such as protamine is administered for the purpose of hemostasis.

Thereafter, the tools are removed (step S13). Specifically, the balloon catheters 32, 33, and 34, the non-branched sheath 40, the three-way sheath 70, and tourniquets 35 are removed, and a hemostatic procedure is performed. When the patient is awaken from anesthesia, the process is finished.

The perfusion method of the present invention will next be described by way of an example. In this example, a living body to be treated has a tumor in the paraaortic lymph nodes metastasized from the cervical carcinoma. The non-branched sheaths 40-1, 40-2, and 40-3 employed in the example have sizes of 6 Fr., 6 Fr., and 9 Fr., respectively. The three-way sheath 70 has a size of 12 Fr. The balloon catheters 32-1, 32-2, 32-3, 32-4, and 32-5 have sizes of 6 Fr., 6 Fr., 7 Fr., 4 Fr., and 4 Fr., respectively.

In the process of perfusion A, the venous blood is removed through use of cannulas 121 and 122 (360 ml/min), oxygenated in an artificial lung so that the resultant blood has an oxygen saturation level of 99 to 100%, and then is delivered to a feeding tube. In this example, the oxygenated blood was found to have an oxygen saturation level of 99%. The process of perfusion A was continued until the blocking of blood vessels was released after completion of the process of perfusion B described below.

In the process of perfusion B, an anticancer agent cisplatin (140 mg) mixed with saline was fed to the closed region in the aorta through the passage 45 of the non-branched sheath 40-3 at a feed rate of 200 mL/min (liquid removal rate was 190 mL/min). Perfusion B was performed for 30 minutes. During the process of perfusion B, the anticancer agent was quantified by measuring the amount of platinum contained in the blood collected from the predetermined blood vessels; i.e., (i) the abdominal aorta within the blood vessel closed region, (ii) the inferior vena cava within the blood vessel closed region, and (iii) the radial artery outside the blood vessel closed region.

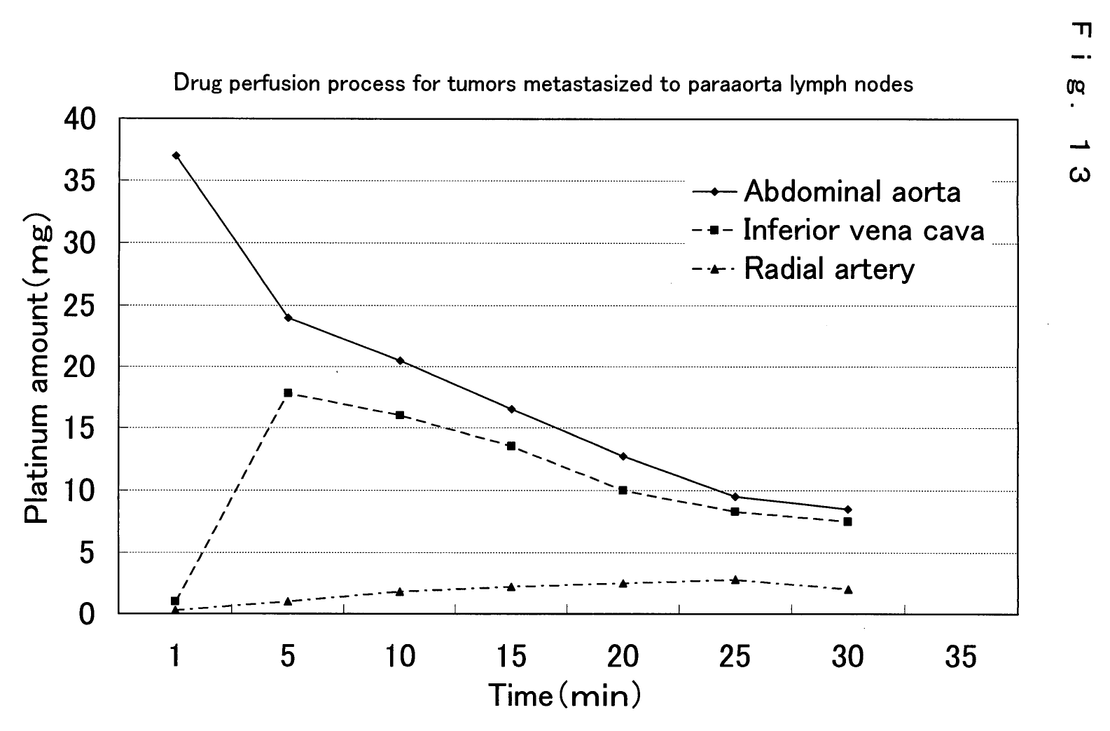

FIG. 13 shows some data obtained in the above experiment. As shown in FIG. 13, in the abdominal aorta within the blood vessel closed region (i) and the inferior vena cava within the blood vessel closed region (ii), cisplatin level was found to be high immediately after administration and then decreased gradually. In contrast, in the radial artery outside the blood vessel closed region (iii) (that is, systemically), substantially no increase in the cisplatin level was observed. These results indicate that the perfusion method of the present invention enables delivery of a drug of high concentration to the blood vessel closed region with substantially no leakage of the drug to outside the blood vessel closed region.

What is claimed is:

1. A drug perfusion method of delivering a drug by way of perfusion to a site of tumor tissue located in a paraaortic lymph node and recovering the drug, comprising:
a perfusion preparatory step including introducing a plurality of balloon catheters into a living body percutaneously so as to indwell the catheters in blood vessels which are branched from the aorta and are present around paraaortic lymph nodes, so that balloon inflation regions of the balloon catheters are positioned at predetermined sites of the blood vessels, and applying to predetermined positions of the lower limbs tourniquets having inflatable regions;
a closed-region creating step of creating a closed region of arteriovenous vessels by inflating the balloons of the balloon catheters and the inflatable regions of the tourniquets so as to cut blood flow to and from the closed region;
an oxygenated blood supply step of feeding oxygenated blood to organs to which blood supply has been cut as a result of the closed-region creating step having been performed;
a drug administration step of administering a drug solution containing a drug to the closed region; and
a bodily fluid recovering step of recovering from the closed region bodily fluid containing the drug.

2. The drug perfusion method as recited in claim 1, wherein the balloons of the balloon catheters are indwelled so that the balloon inflation regions are positioned at predetermined sites in at least the right renal artery, the left renal artery, the celiac artery, the superior mesenteric artery, the inferior mesenteric artery, the aorta proximal to the heart with respect to the branching portion of the celiac artery, and the inferior vena cava.

3. The perfusion method as recited in claim 2, wherein oxygenated blood is fed to organs through a balloon catheter indwelled in the right renal artery, a balloon catheter indwelled in the left renal artery, and at least one balloon catheter selected from among the three balloon catheters indwelled at predetermined positions in the celiac artery, the superior mesenteric artery, and the inferior mesenteric artery.

4. The drug perfusion method as recited in claim 2, wherein a drug is administered through a balloon catheter which is indwelled in the aorta at a predetermined position thereof, the position being on the heart side with respect to the branching portion of the celiac artery.

5. A catheter-introducing sheath for introducing into a living body a catheter which is used for performing the perfusion method of delivering, by way of perfusion, a drug to a paraaortic lymph node tumor, comprising:
a first passage for allowing passage of a first catheter therethrough, a second passage branching from the first passage and allowing passage of a second catheter therethrough, and a third passage branching from the first passage and allowing passage of a third catheter therethrough.

6. A catheter-introducing sheath for introducing into a living body a catheter which is used for performing the perfusion method of delivering, by way of perfusion, a drug to a paraaortic lymph node tumor, comprising:
a tubular body having a first passage for allowing passage of a catheter therethrough,
a first branched connector which is connected to an end of the tubular body and which has a second passage and a third passage, the second passage directly communicating, via a first end of the first branched connector, with the first passage and the third passage branching from the second passage, and
a second branched connector which is connected to a second end of the first branched connector, the second end being opposite the first end, and which has a fourth passage and a fifth passage, the fourth passage directly communicating with the second passage and the fifth passage branching from the fourth passage.

7. The catheter-introducing sheath as recited in claim 6, wherein the first and the second branched connectors are made of the same material.

8. The catheter-introducing sheath as recited in claim 6, wherein the axis running through the first, second, and the fourth passages, the axis running through the third passage, and the axis running through the fifth passage do not lie within a single plane.

9. A catheter introducer for introducing into a living body a catheter-inserting sheath which is used for performing the perfusion method of delivering, by way of perfusion, a drug to a paraaortic lymph node tumor, comprising:
a catheter-inserting sheath, and
a dilator;
wherein the catheter-inserting sheath comprises:
a first passage for allowing passage of the first catheter therethrough,
a second passage branching from the first passage and allowing the second catheter to pass therethrough, and
a third passage branching from the first passage and allowing the third catheter to pass therethrough.

10. A catheter introducer for introducing into a living body a catheter-inserting sheath which is used for performing the perfusion method of delivering, by way of perfusion, a drug to a paraaortic lymph node tumor, comprising:
a catheter-inserting sheath, and
a dilator;
wherein the catheter-inserting sheath comprises:
a tubular body having a first passage for allowing passage of a catheter therethrough,
a first branched connector which is connected to an end of the tubular body and which has a second passage and a third passage, the second passage directly communicating, via a first end of the first branched connector, with the first passage and the third passage branching from the second passage, and
a second branched connector which is connected to a second end of the first branched connector, the second end being opposite the first end, and which has a fourth passage and a fifth passage, the fourth passage directly communicating with the second passage and the fifth passage branching from the fourth passage.

11. The catheter introducer as recited in claim 9, wherein the dilator is inserted into the first passage.

12. The catheter introducer as recited in claim 9, wherein the dilator is inserted into a passage formed by the first, second, and the third passage.

13. An oxygenated blood perfusion apparatus equipped with fluid discharge means for discharging bodily fluid out of a living body, bodily-fluid-oxygenating means for oxygenating the discharged bodily fluid, and feeding means for feeding oxygenated blood into the living body through a plurality of balloon catheters indwelled in the living body, wherein any one to three of said plurality of balloon catheters are respectively inserted into the living body through their corresponding catheter-inserting sheaths, each sheath comprising:
a first passage for allowing passage of the first catheter therethrough,
a second passage branching from the first passage and allowing the second catheter to pass therethrough, and
a third passage branching from the first passage and allowing the third catheter to pass therethrough.

14. An oxygenated blood perfusion apparatus equipped with fluid discharge means for discharging bodily fluid out of a living body, bodily-fluid-oxygenating means for oxygenating the discharged bodily fluid, and feeding means for feeding oxygenated blood into the living body through a plurality of balloon catheters indwelled in the living body, wherein one to three of said plurality of balloon catheters are respectively inserted into the living body through their corresponding catheter-inserting sheaths, each sheath comprising:
a tubular body having a first passage for allowing passage of a catheter therethrough,
a first branched connector which is connected to an end of the tubular body and which has a second passage and a third passage, the second passage directly communicating, via a first end of the first branched connector, with the first passage and the third passage branching from the second passage, and
a second branched connector which is connected to a second end of the first branched connector, the second end being opposite the first end, and which has a fourth passage and a fifth passage, the fourth passage directly communicating with the second passage and the fifth passage branching from the fourth passage.
